(19) United States
(12) Patent Application Publication
(10) Pub. No.: US 2026/0027308 A1
(43) Pub. Date: Jan. 29, 2026

(54) AUTOMATED AND SEMI-AUTOMATED MIXING AND AUTOMATED DELIVERY AUTOINJECTOR

(71) Applicant: Windgap Medical, Inc., Watertown, MA (US)

(72) Inventors: Jeffrey Thomas Chagnon, Somerville, MA (US); Andrew Ryan, Militon, MA (US); Jameson Woods, Watertown, MA (US); Tali Kurlantzick, Newton, MA (US); Daniel Yasevac, Needham, MA (US)

(21) Appl. No.: 19/350,496

(22) Filed: Oct. 6, 2025

Related U.S. Application Data
(63) Continuation of application No. PCT/US2024/023643, filed on Apr. 8, 2024.
(60) Provisional application No. 63/457,585, filed on Apr. 6, 2023.
(60) Provisional application No. 63/457,567, filed on Apr. 6, 2023.

(51) Int. Cl. A61M 5/32; A61J 1/20; A61J 1/22; A61M 5/19; A61M 5/20

(52) U.S. Cl. CPC A61M 5/3294; A61J 1/201; A61J 1/2037; A61J 1/2096; A61J 1/22; A61M 5/19; A61M 5/2046; A61M 5/2066; A61M 2205/3317

(57) ABSTRACT

An automated, and/or semi-automated, reciprocating medicament mixing and injector system, where the energy provided to transfer medicament components back and forth between containers or cartridges can be redirected to deliver the mixed medicament components. A pressurized gas source can drive the flow through a plurality of valves to mix medicament between containers. In a fully automated embodiment, a user activated button advances a magnet to power solenoid valves to control mixing, and then dispensing the medicament. In a semi-automated embodiment, a user activated button releases pressurized gas, and mixing button routes the gas through alternating valves fluidly coupled to the containers to mix the medicaments.

### CROSS REFERENCE TO RELATED APPLICATIONS

This application is a Continuation of International Application No. PCT/US24/23643, filed on Apr. 8, 2024, which claims the benefit of priority to U.S. Provisional No. 63/457,585, filed on Apr. 6, 2023, and U.S. Provisional No. 63/457,567, filed on Apr. 6, 2023; the entire content of each is hereby incorporated by reference.

### BACKGROUND OF THE DISCLOSED SUBJECT MATTER

### Field of the Disclosed Subject Matter

The present invention relates generally to dual container devices for reconstituting or mixing medicament components. In addition to working well for large volumes of active pharmaceutical ingredient(s) and diluent that require mixing, the devices disclosed herein are particularly useful for difficult to mix drugs. This difficulty could come in the form of high viscosity requiring a large amount of force, or an API that requires a large amount of agitation in order to evenly mix into the diluent. The devices disclosed herein can scale or be sized to accommodate standard drug cartridges, e.g. from 1 mL to 5 mL in volume each but cartridges less than 1 mL and larger than 5 mL are also contemplated.

### Description of Related Art

Dual container/cartridge injector/autoinjectors are known for storing drug components separately until reconstitution or mixing at point of use. There are various benefits to therapeutics which may be preferred to be provided in a multi-chamber format. The drug may be less thermally stable, have a shorter shelf life, or have other issues being in its aqueous form. Solubilizing drugs in liquid agents, suspending dry particles in liquids, or combining liquid-liquid solutions or suspensions thereof may be required for similar reasons.

In the field of use of multi-chambered injector/autoinjectors, there are also drug formulations where high-intensity and/or longer duration mixing is needed after recombination of the drug constituents prior to delivery of difficult to mix drug products. This may be due to low solubility of the drug, poor surface energy or wettability of a powder or microparticle for dissolution. Other needs include making a suspension of particles homogeneously dispersed within a solvent, solving problems with caking of a dry phase requiring initial energy for dispersion, or poor miscibility making emulsification difficult. In some cases, speed and ease-of-use may be critical for rescue applications where an emergency treatment needs to be delivered very quickly and with very few steps. In this field of use, state-of-the-art devices typically rely on a user shaking the drug container to mix, dissolve, or suspend the drug. Preparation can also require multiple steps that include changing out needles, or moving drug and diluent from one container to another manually. As a result of these additional user-required step, users may experience: delays in treatment time, inadequately mixed drugs, or become generally dissatisfied with the experience of using the product. In other cases, drugs may be formulated in less ideal ways where users may be required to inject a higher dose volume, endure a less comfortable dosage form, a larger than desirable delivery needle, be exposed to additional solubilizing or stabilizing agents added to the formulation, or be required to make more frequent injections. There is significant motivation to create a device that can improve upon the mixing of drugs which are otherwise difficult to solubilize, reconstitute, or suspend by re-combination alone.

The present application seeks to solve some of these identified problems as well as other problems that will become apparent to those skilled in the art.

### SUMMARY OF THE DISCLOSED SUBJECT MATTER

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter includes a mixing and drug delivery system comprising: a housing configured to hold a first container and a second container, wherein the first container contains a first medicament component and the second container contains a second medicament component; a first seal associated with the first container; a second seal associated with the second container; a fluid communication assembly having a fluidic channel between the first container and the second container, the fluid communication assembly configured to be displaced from a first position to a second position within the housing thereby opening, removing or otherwise piercing the first seal and second seal to provide a fluidic pathway between the first container and the second container; a mixing system configured to alternately transfer the first and second medicaments between the first and second containers during a mixing phase; a pressurized gas chamber at least partially disposed in the housing to pressurize the mixing system; a mixing activation mechanism, the mixing activation mechanism displacing the pressurized gas chamber from a first position to a second position to open or otherwise pierce the pressurized gas chamber; at least one valve configured to release a portion of pressurized gas that facilitates the transfer of the first and second medicaments components between the first and second containers by the mixing system, wherein the transfer between first and second containers causes the first and second medicament components to become a mixed medicament; and a needle delivery assembly configured to be in fluid communication with the first and second containers during a delivery phase.

In some embodiments, the mixing activation mechanism is coupled to a power source, the power source displaced from a first position to a second position to activate the at least one valve.

In some embodiments, the power source controls operation of the at least one valve.

In some embodiments, the power source is a magnet.

In some embodiments, the at least one valve includes two solenoid valves, a first solenoid valve in fluid communication with the first container and a second solenoid valve in fluid communication with the second container.

In some embodiments, only one solenoid valve is powered at a time.

In some embodiments, the first and second solenoid valves are powered simultaneously.

In some embodiments, the first and second solenoid valves are initially in a closed position.

In some embodiments, the pressurized gas chamber is disposed above the first container and a second container.

In some embodiments, the mixing system further comprises a first gas-driven plunger associated with the first container and a second gas-driven plunger associated with the second container.

In accordance with another aspect of the disclosure, a mixing and drug delivery system is provided which comprises: a housing configured to hold a first container and a second container, wherein the first container contains a first medicament component and the second container contains a second medicament component; a first seal associated with the first container; a second seal associated with the second container; a fluid communication assembly having a fluidic channel between the first container and the second container, the fluid communication assembly configured to be displaced from a first position to a second position within the housing thereby opening, removing or otherwise piercing the first seal and second seal to provide a fluidic pathway between the first container and the second container; a mixing system configured to alternately transfer the first and second medicaments between the first and second containers during a mixing phase; a pressurized gas chamber at least partially disposed in the housing to pressurize the mixing system; a mixing activation mechanism, the mixing activation mechanism displacing the pressurized gas chamber from a first position to a second position to open or otherwise pierce the pressurized gas chamber; at least one valve configured to release a portion of pressurized gas that facilitates the transfer of the first and second medicaments components between the first and second containers by the mixing system, wherein the transfer between first and second containers causes the first and second medicament components to become a mixed medicament; a mixing button, the mixing button movable between a first position and a second position to open and close the at least one valve; and a needle delivery assembly configured to be in fluid communication with the first and second containers during a delivery phase.

In some embodiments, the mixing system further comprises a first fluid path from the pressurized gas chamber through the valve to the first container, and a second fluid path from the pressurized gas chamber through the valve to the second container.

In some embodiments, the mixing system further comprises a first gas-driven plunger associated with the first container and a second gas-driven plunger associated with the second container.

In some embodiments, the at least one valve includes a multi-directional valve configured to alternate the flow of gas directed to the first and second gas-driven plungers based on user input to the mixing button.

In some embodiments, whereupon receiving the user input to the mixing button causes the mixing system to drive the first gas-driven plunger to transfer the first medicament component from the first container into the second container with the second medicament component.

In some embodiments, displacement of the first gas-driven plunger downward in the first container causes displacement of the second gas-driven plunger upwards in the second container.

In some embodiments, a valve release component is coupled to the multi-directional valve, valve release component displaced from a first position to a second position within the housing.

In some embodiments, pressurized gas flows to only one of the first plunger or second plunger at a time when the valve release component is in the first position.

In some embodiments, pressurized gas flows to both the first plunger and the second plunger simultaneously when the valve release component is in the second position.

In some embodiments, a delivery seal is configured to prevent fluid communication between the fluidic channel and the needle during a mixing phase.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the method and system of the disclosed subject matter. Together with the description, the drawings serve to explain the principles of the disclosed subject matter.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIGS. 1-3 are schematic representations of the drug cartridges, cartridge holder and mixing assembly in accordance with the disclosed subject matter. The exemplary embodiments illustrated can be incorporated into both a fully automated mixing and delivery autoinjector, and a semi-automated mixing and delivery autoinjector.

FIGS. 4-5 are schematic representations of an external view of the fully automated mixing and delivery autoinjector.

FIGS. 6-9 are schematic representations of an activation assembly of the fully automated mixing and delivery autoinjector.

FIGS. 10-11 are schematic representations of a drug mixing process of the fully automated mixing and delivery autoinjector.

FIGS. 12-16 are schematic representations of a drug delivery process of the fully automated mixing and delivery autoinjector.

FIG. 17 is a schematic representation of the optional electronic subcomponents that enable indicators to the user on the status of the mixing and delivery process.

FIGS. 18-19 are schematic representations of an external view of the semi-automated mixing and delivery autoinjector.

FIGS. 20-24 are schematic representations of an activation assembly of the semi-automated mixing and delivery autoinjector.

FIGS. 25-27 are schematic representations of a drug mixing process of the semi-automated mixing and delivery autoinjector.

FIGS. 28-33 are schematic representations of a drug delivery process of the semi-automated mixing and delivery autoinjector.

FIGS. 34-35 are schematic representations of a thread captured gas cannister for coupling with the pressure regulator.

FIGS. 36-37 are schematic representations of a cap captured gas cannister for coupling with the pressure regulator.

### DETAILED DESCRIPTION OF AN EXEMPLARY EMBODIMENT

Reference will now be made in detail to exemplary embodiments of the disclosed subject matter, an example of which is illustrated in the accompanying drawings. The method and corresponding steps of the disclosed subject matter will be described in conjunction with the detailed description of the system.

To provide clarity, the applicants would like to provide context around certain terms used throughout this description that is in addition to their ordinary meaning.

Distal or distal end primarily refers to the end of the mixing and injector system having the components and features to drive the plungers. In contrast, proximal or proximal end refers to the end of the device where the plungers are being driven into. For example, in all of the embodiments disclosed the delivery needle is disposed on the proximal end of the mixing and injector systems. Additionally, the distal end of the delivery needle is the end that is receiving the mixed medicament components, whereas the proximal end of the delivery needle is injecting the mixed medicament components into a recipient or otherwise releasing the mixed medicament components.

For purposes of this application the term container can include any component that is configured to hold a volume. For example, a cartridge, pre-filled syringe, a vial and so forth would be considered a container. Containers can have attachment points, removable or pierceable seals associated with them and have medicament components stored therein.

As noted, there is a need to improve upon drug mixing devices to allow for drug formulations where high-intensity and/or long duration mixing is needed after combination of the drug constituents. The inventors, who created the embodiments herein, have provided solutions to at least this noted problem as well as other problems that will become apparent upon reading this description.

In many of the embodiments provided herein there is provided a fluid communication system, that includes a pair of mixing needles, a fluidic channel and a frame. This system can be positioned in the housing in a fixed manner, where other systems engage into it, or it can movable in a distal and/or proximal manner to engage with the containers as well as needle delivery system. Greater detail and examples of this fluid communication system can be found in U.S. published application US2022/0001112 A1, US2022/0379033, and/or US 2022/0001112, each of which is hereby incorporated by reference in its entirety.

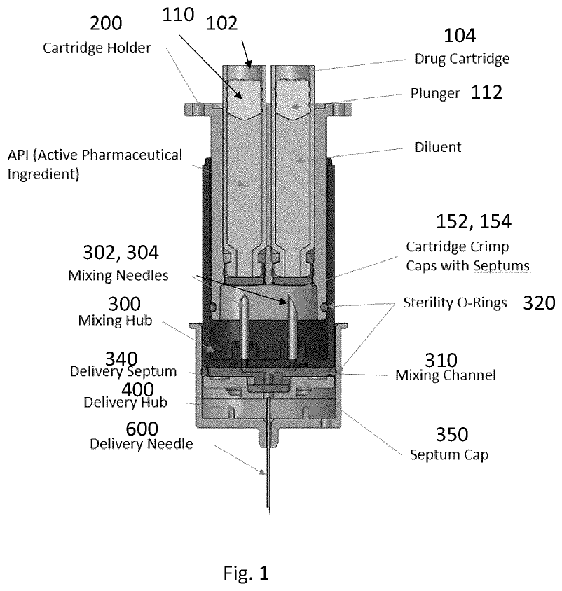

For the purpose of explanation and illustration, and not limitation, exemplary embodiments of the system in accordance with the disclosed subject matter is shown in FIG. 1 and is designated generally by reference character 1000. Similar reference numerals (differentiated by the leading numeral) may be provided among the various views and Figures presented herein to denote functionally corresponding, but not necessarily identical structures.

The methods and systems presented herein may be used for large volume dual chamber (LVDC) primary drug container (PDC) which is used to facilitate storage, mixing, and delivery of a pharmaceutical ingredient and diluent. The diluent and Active Pharmaceutical Ingredient (API) are held in separate drug cartridges 102, 104 within the device. In an exemplary embodiment, the PDC scales to accommodate standard drug cartridges from about 1 mL to about 5 mL in volume, each. It is also contemplated in another embodiment (not shown) that the cartridges could be prefilled syringes.

The drug cartridges 102, 104 are held in the cartridge holder 200 to prevent them from moving during storage or use. The cartridges can be held via “interference-fit” or friction with adjacent structures/surfaces of the housing, and/or via mating engagement (e.g. mechanical interlock such as male/female complimentary surface features) to retain a fixed orientation with respect to the cartridge holder.

The cartridge holder 200 fits within the mixing hub 300 such that the two components can be displaced (e.g. slide or translate vertically) relative to each other, but the walls of the mixing hub 300 help maintain a specific orientation of the cartridge holder. For example, the upwardly extending walls of the mixing hub circumscribe at least a portion of the cartridge holder 200, thereby orienting the two components to have aligned central axes. In some embodiments, the mixing hub 300 contains at least one (e.g. two equidistantly spaced) needles affixed 302, 304 (e.g. adhesively attached, insert molded, or integrally formed) to the base of the component. Each needle can be positioned below a central longitudinal axis of the drug cartridges 102, 104. Corresponding through holes concentric to the needles 302, 304 are included in the bottom of the mixing hub, such that anything that flows through the needles can flow through the mixing hub 300 as well. Thus, these components form a fluid communication assembly of the cartridges 102, 104. In the embodiment with the prefilled syringe, alternative to the cartridge-based design, the septums 152, 154 would swap positions with the mixing needles 302, 304. That is, the mixing needles 302, 304 would be directly staked into the drug cartridges 102, 104 (which can be made of glass or plastic) with the seals 152, 154 affixed and attached to the inlets of the mixing channel.

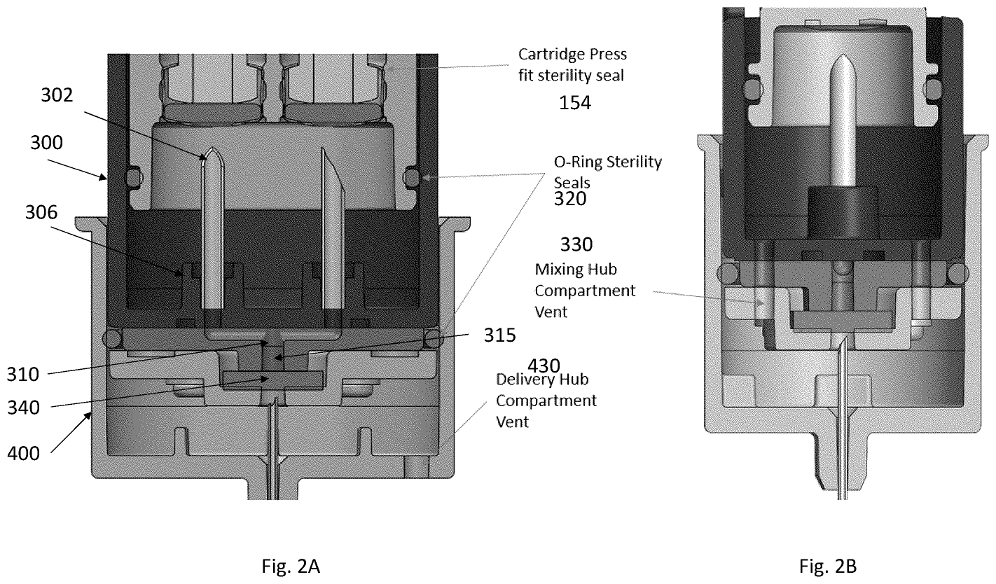

In some embodiments, as best shown in FIG. 2a, the mixing needles 302, 304 can be seated within upwardly extending needle receptacles 306 in the base of the missing hub, sized with an inner diameter sufficient to receive the outer diameter of the needles 302, 304 therein. The bottom face of the mixing hub 300 contains a plurality (e.g. four equidistantly spaced) posts that help align and secure both the mixing channel 310 and the septum cap 450. Thread forming screws are screwed into the posts to secure the mixing channel 310 and septum cap 350 to the base of the mixing hub 300.

The mixing channel 310 contains a groove that connects the mixing needles 302, 304 in the mixing hub 300. This groove allows fluid and air to flow or transfer between the two needles, and thus into and out of the two drug cartridges 102, 104 (when unsealed) when an external force is applied to the plungers 110, 112 within the drug cartridges. To ensure that no fluid escapes from this groove of the mixing channel 310, there is a second concentric groove that surrounds the central groove. This groove contains an O-Ring 320 that is compressed between the mixing channel 310 and mixing hub 300 creating a seal.

Additionally, a through hole 315 is included in fluid communication with and disposed below the mixing channel 310 which serves two functions: 1) for fluid to pass between the two mixing needles; and 2) for fluid to exit out of the channel (and downwardly into the delivery needle). In the exemplary embodiment shown, the through hole is located at the center of the device, equidistantly spaced between the two needles 302, 304, and vertically aligned with the delivery needle 600. At the end of the through hole is a septum 340 (which can be formed of a resilient elastomeric member) that is compressed against the mixing channel 310 by the septum cap 350. The septum seals the through hole of the mixing channel 310 (until being pierced or opened by the delivery needle 600, as described below).

The mixing hub 300 along with mixing channel 310, and septum cap 350 fit within the delivery hub 400. Similar to the mixing hub 300 and cartridge holder 200, The delivery hub's upwardly extending walls help to guide the other components such that they can slide relative to each other with a specific orientation. In the exemplary embodiment the delivery hub 400 has upwardly extending sidewall that circumscribes at least a portion of the mixing hub 300 received therein; and the mixing hug 300 in turn receives the cartridge holder 200 therein (which contains the drug cartridges 102, 104), as described above. Thus, the device can be configured with a nesting arrangement, in cascading order, of: the drug cartridges, cartridge holder, mixing hub and delivery hub.

Additionally, the delivery hub 400 has the delivery needle 600 affixed (e.g. glued, insert molded, or affixed in some other fashion) into its base that is used for delivery of the mixed active pharmaceutical ingredient(s) to its intended target. The delivery needle 600 can be located at the center of the delivery hub and extend both upwardly into the interior of the delivery hub 400, and downwardly beyond the lower boss on the bottom surface of the delivery hub 400. In another embodiment, the delivery needle is not located at the center of the delivery hub but offset by some amount.

### Sterility Features

The device disclosed herein contains many features that are specially used for sterility purposes. When stored, the primary drug container prevents ingress of particles and bacteria from reaching critical interfaces that could introduce such bacteria to the patient.

With reference to FIGS. 2A-FIG. 2B, the first main area to ensure sterility is the mixing hub compartment 300 created by the void in space between the bottom of the cartridge holder 200 and the inner surfaces of the mixing hub 300. Seals are created to ensure that no particulate reaches the mixing needles, or tops of the drug cartridges. The cartridges 102, 104 are press fit into the cartridge holder 200 to create a radial seal that no particulate can bypass.

In some embodiments, a seal is established between the cartridge holder 200 and mixing hub 300. For purpose of illustration and not limitation, in an exemplary embodiment an O-Ring groove 319, with O-Ring 320 disposed therein, along the outer wall of the cartridge holder 200 creates a seal between the cartridge holder 200 and the mixing hub 300. A hole in the bottom of the mixing hub 300 establishes a mixing hub compartment vent 330. This vent is initially covered by a piece of cover or film (e.g. Tyvec™) that is ultrasonically welded to the plastic to create a seal. The O-Ring seal 320 between mixing channel 310 and the mixing hub 300 and the septum 340 seal between the mixing channel 310 and septum caps 350 that were described earlier are the last sealing surfaces that ensure this compartment is completely sealed off.

The second compartment is the delivery hub compartment created by the mixing hub 300 and the inner walls of the delivery hub 400. The mixing channel 310 and the septum cap 350 are of similar shape, but the mixing channel is slightly smaller which creates an O-Ring groove. The outer surface of the mixing channel 310 supports the inner diameter of the O-Ring, while the lower and top surfaces of the mixing hub 300 and septum cap 350 respectively support the O-Ring 320 from moving. The delivery needle 600 is affixed (e.g. glued) into the delivery hub which prevents particulate from bypassing along the exterior surface of the needle.

The delivery hub 400 has a hole in its lower surface establishing the delivery hub compartment vent 430. This Vent is initially covered by a cover or film (e.g. Tyvec™) that could be ultrasonically welded to the plastic to create a seal. The intent of the lower surface of the delivery hub is that a safety cap fits over the needle 600 and press fit around the lower boss that the delivery needle 600 protrudes from. This creates the final seal to enclose the delivery hub compartment 400 and delivery needle 600 and ensure all remain sterile prior to use.

### Device States of Operation

The device disclosed herein has a plurality (e.g. three) different device states throughout its operation life cycle: Nominal, Mixing, and Delivery. In the nominal state, the components are all assembled together as described above in connection with FIGS. 1-2C, and the different compartment are all sealed and sterile.

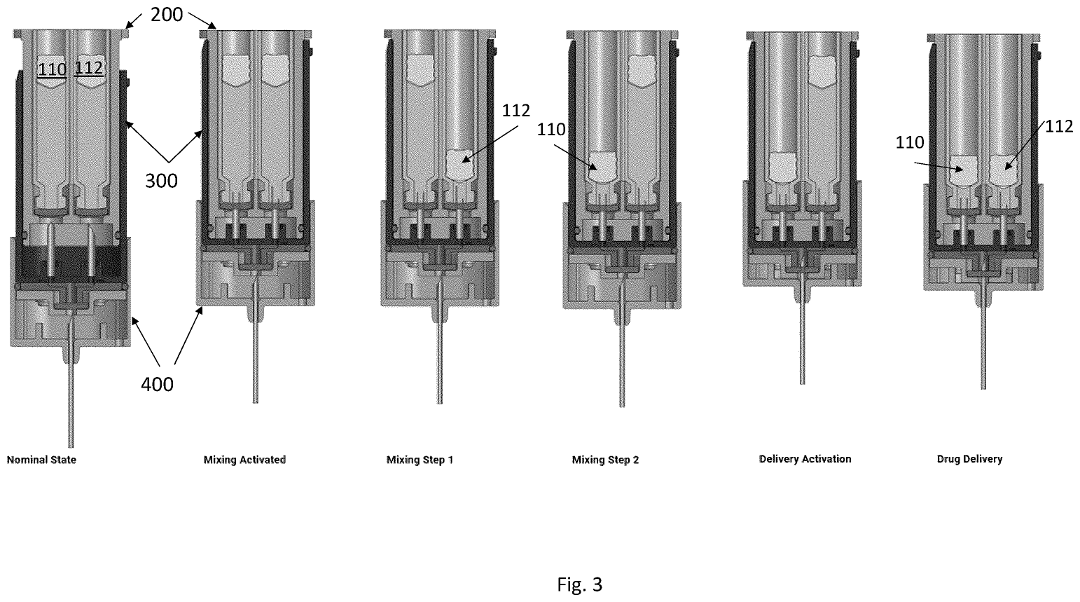

The device remains as shown in FIG. 1 (and as shown in the first illustration in FIG. 3) until the cartridge holder 200 slides down relative to the Mixing Hub 300 causing the mixing needles 302, 304 to pierce the drug cartridges 102, 104 and open a fluid pathway between the two cartridges (as shown in the second illustration in FIG. 3). The cartridge holder 200 continues to slide until the lower face contacts the inner/upper surface of the mixing hub 300 to prevent additional downward motion of the cartridge holder 200.

Once the fluid pathway is opened between the two cartridges 102, 104 in the Mixing State, the drug can be mixed by transferring from one cartridge, through the mixing channel 310, and into the adjacent cartridge. For example, this is completed by pushing on the right plunger 112 to push the diluent through the fluid pathway 310 and into the opposing drug cartridge 102 (as shown in the third illustration in FIG. 3). Once the right plunger 112 reaches the bottom of the drug cartridge the force or pressure used is released. Next the same force or pressure is applied to the left plunger 110 to push the partial mixture through the mixing channel 310 and into the right drug cartridge 104 (as shown in the fourth illustration in FIG. 3). This force remains until the left plunger 110 reaches the bottom of the cartridge 102. This process is repeated for a desired number of cycles until the diluent and API are fully mixed.

While this exemplary embodiment describes a full depression of each plunger over the entire range of motion to the distal end of the containers, only a partial depression of either (or both) plungers is within the scope of the present disclosure. Thus any desired amount of diluent, or API, can be transferred from one drug cartridge to another.

Once the desired amount of mixing is complete, the delivery hub 300 moves up relative to the rest of the assembly. This causes the proximal, or non patient end, of the delivery needle 600 to pierce the septum 340 and enter the mixing channel 310 (as shown in the fifth illustration in FIG. 3). The delivery hub 400 continues to move until the component contacts the septum cap 350 and motion is stopped. To deliver the API through the delivery needle 600, a force or pressure is applied to the back of both plungers 110, 112 in the drug cartridges to push the mixture through the mixing channel and into the delivery needle (as shown in the sixth illustration in FIG. 3).

In accordance with an aspect of the disclosure, the automatic mixing and automatic delivery (AMCAD) autoinjector is built around the large volume dual chamber primary drug container configuration to allow a user to mix and deliver viscous drugs. The user controls the activation and point of delivery, but the mixing process and delivery force is controlled by the device. This design removes as many user steps as possible to ensure that the mixing performance would not be affected by the user.

### Device Use States

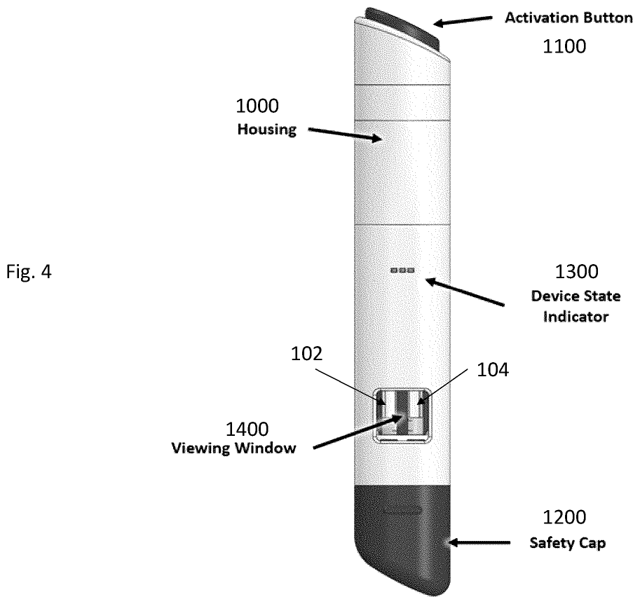

In the exemplary embodiment shown in FIG. 4, the device contains a housing that serves as the main body for the user to hold during device use. The primary touch points for this device are the activation button 1100 located at the top of the housing (though alternative locations, e.g. side, are within the scope of this disclosure) and the safety cap 1200, which encloses the delivery needle 600, that is removed prior to delivery. There are also a plurality of locations (e.g. two) where the user can obtain information about the device state. these are the device state indicator 1300 that contains several indicia (e.g. in one embodiment, three LEDs) and the Viewing Window 1400 that allows the user to see into the interior drug cartridges and evaluate the mixture status and dispensing operation.

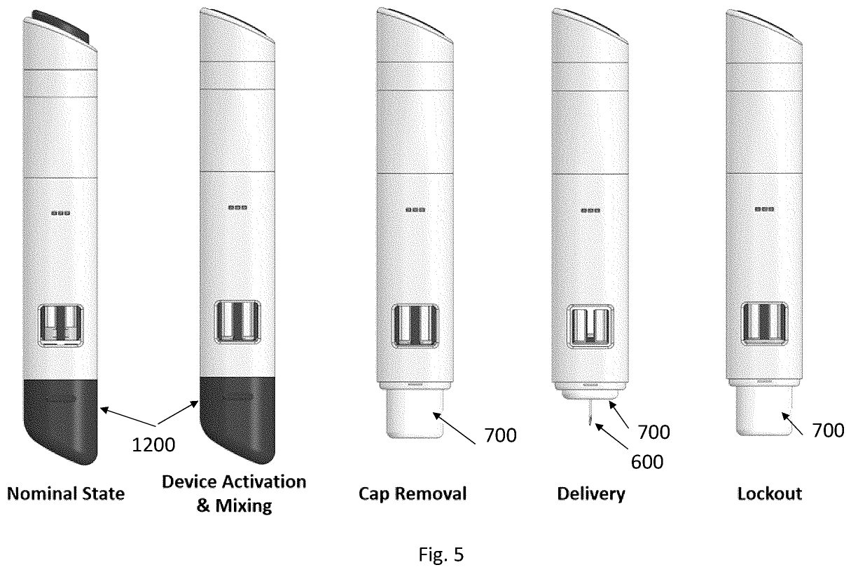

The AMCAD device is designed to minimize the total number of use case steps for the user. When the user removes the device from its packaging, the device is in its nominal state, as shown in FIG. 5. There is no power to the device, the activation button extends beyond the outer/upper surface of the housing, and the safety cap is securely attached. To activate the device and begin the mixing process, the user depresses the activation button 1100. Once this is done the device turns on and completes the mixing process. Once the mixing process is completed, the user removes the safety cap 1200 to expose the needle shield 700. The user places the needle shield 700 up against the injection site on the patient, and presses the device into the patient. This causes the delivery needle 600 in the primary drug container to pierce the skin and open the delivery fluid pathway. Once the delivery is complete the user can pull the device away from the patient and the needle shield 700 will extend and lockout.

### Device Activation

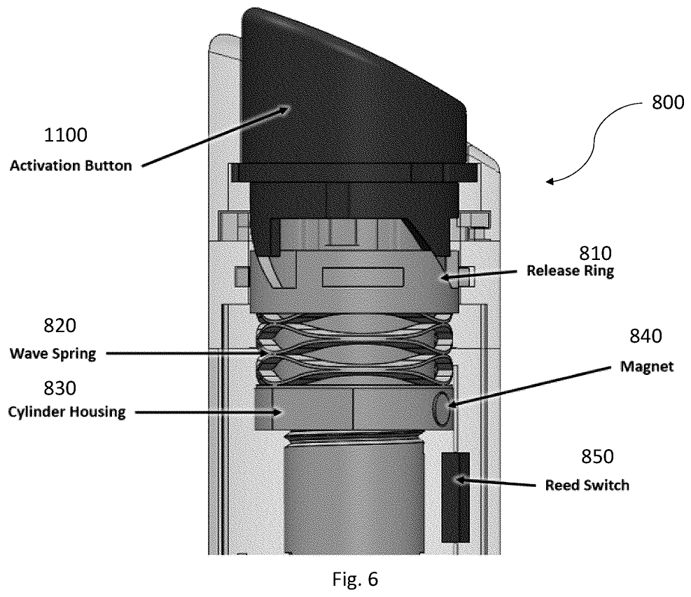

In the exemplary embodiment shown in FIG. 6, the device activation mechanism 800 includes an activation button 1100, a release ring 810, an actuator with stored energy (e.g. wave spring) 820, and cylinder housing 830 wherein the actuator 820 is biased to exert a force on the cylinder housing 830 to displace the cylinder housing downwardly. Although the exemplary embodiment illustrates a wave spring, additional or alternative mechanisms can be employed to exert the requisite force on the cylinder housing. The release ring 810 is held in place vertically by features on the housing but is free to rotate about the cylinder housing 830. The cylinder housing 830 has grooves (e.g. “L-Shaped”) in its body that features on the release ring 810 interface with. The release ring 810 is used to compress the wave spring 820 against the cylinder housing 830, and then lock in place by rotating into the lower part of the groove(s).

In operation, when the user activates the device by pushing on the activation button 1100, the downwardly projecting ramps 1102 on the activation button are advanced into engagement with corresponding cam surfaces on the release ring 810. The exemplary embodiment shown, the angle of incidence of the ramp and cam surfaces is approximately 45 degrees, though other angles can be employed. The interaction of the ramps and cams causes the release ring 810 to rotate out from the grooves until there is no longer any retention of the cylinder housing 830 with respect to the release ring 810. With the release ring 810 held in place by the housing, the now released, cylinder housing 830 is driven downwards by the spring 820.

In accordance with another aspect of the disclosure, a power source is incorporated into the cylinder housing 830 to activate the mixing, and delivery sequences. A variety of power sources can be employed, and the exemplary embodiment of FIG. 6 depicts a magnet 840 embedded in the cylinder housing 830 is driven downwards to magnetically/electrically engage an electrical switch 850 (e.g. reed switch). This switch provides power to the control board and the rest of the electrical system driving the flow of gases through fluidly coupled valves to generate medicament flow (mixing and dispensing). In the exemplary embodiment the power source 840 is incorporated into a lower flange of the cylinder housing, upon which the bottom of the spring 820 abuts to exert the downward force described above. Initially, the power source 840 is spaced a distance above the switch 850 (in the embodiment of a magnetic power source 840, the distance is large enough such that the magnetic field generated by the magnet 840 does not, when in initial position, induce a current in the switch 850). Upon release of the spring 820, the magnet 840 is displaced downwardly into proximity (e.g. horizontally aligned or partially overlapping) with the switch 850 to induce an electrical signal in the switch and activate the circuit and valves.

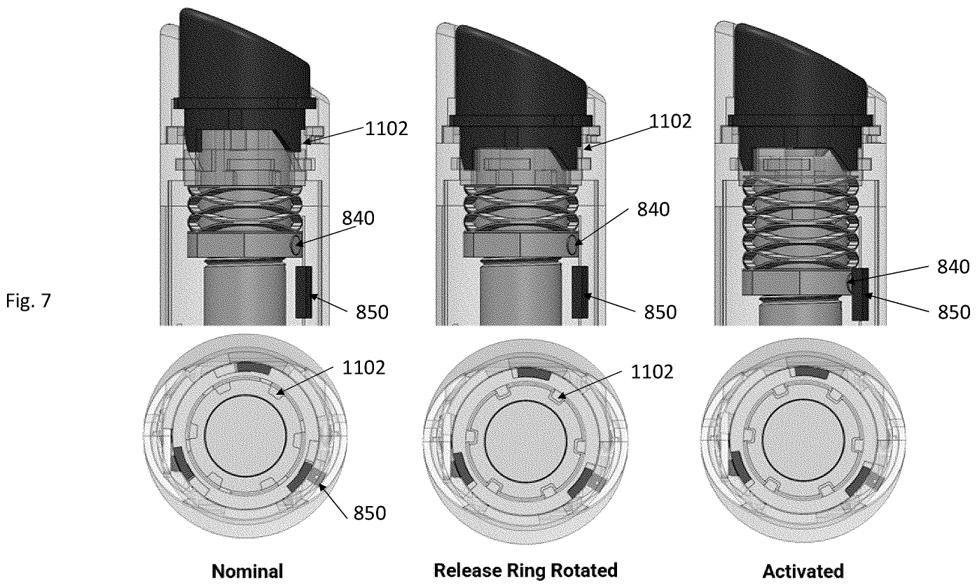

As shown in FIG. 7, in the nominal state, the power source 840 is spaced above the switch 850, and no power is provided to the device. Also, the ramps 1102 of the release ring 830 are circumferentially spaced or offset (as shown in the top views at the bottom of FIG. 7). As the cams and ramps 1102 rotate the release ring, the spring 820 is released and elongates to push the power source 840 down and into engagement with the reed switch. In an exemplary embodiment, the distance the power source travels is approximately 1 mm˜30 mm. Therefore, an initial mechanical force applied by the device activation mechanism establishes an electrical power to activate and operate the mixing and dispensing, as described in further detail below.

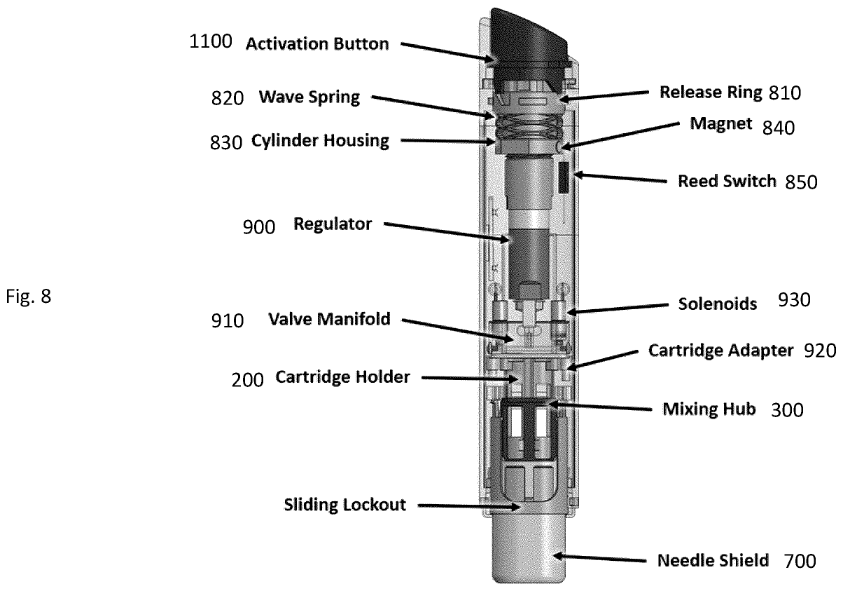

Referring now to FIG. 8, as the cylinder housing 830 is driven downwards by the spring 820, the regulator 900 and gas cannister are driven downwards as they are rigidly attached to the cylinder housing 830. In some embodiments the gas cannister is located above the regulator. In some embodiments the gas cannister can be housed within the regulator. This motion continues until the gas cylinder is pierced and can no longer move relative to the valve manifold 910. In some embodiments the gas canister is configured and pierced with a piercing element as disclosed in U.S. Pat. Pub. No. 2022/037903, the entire contents of which are hereby incorporated by reference. Once this occurs, the valve manifold 910 begins to move downwards which in turn causes the cartridge holder 200 to shift down relative to the mixing hub 300. As described above, this motion causes the mixing needles 302, 304 in the mixing hub 300 to pierce the septums in the drug cartridges 102, 104 and open the fluid pathway between the two parallel cartridges.

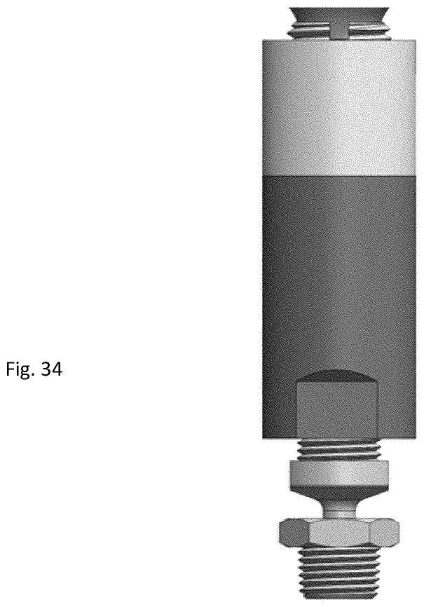

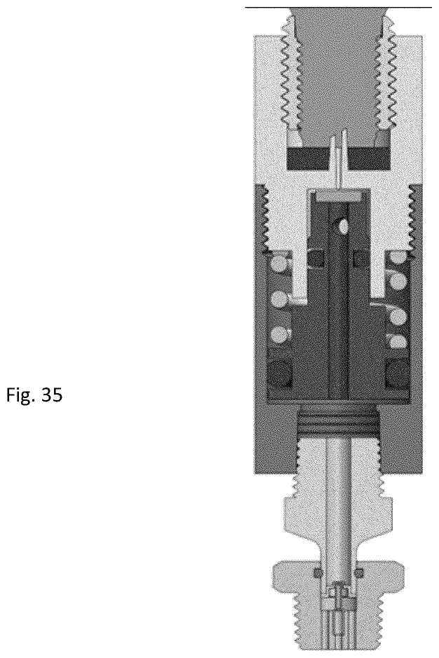

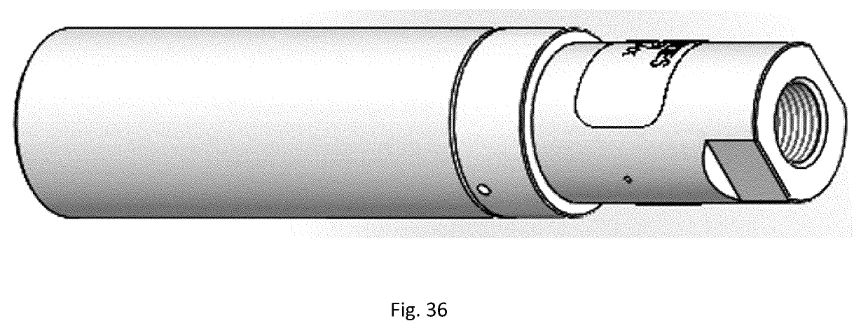

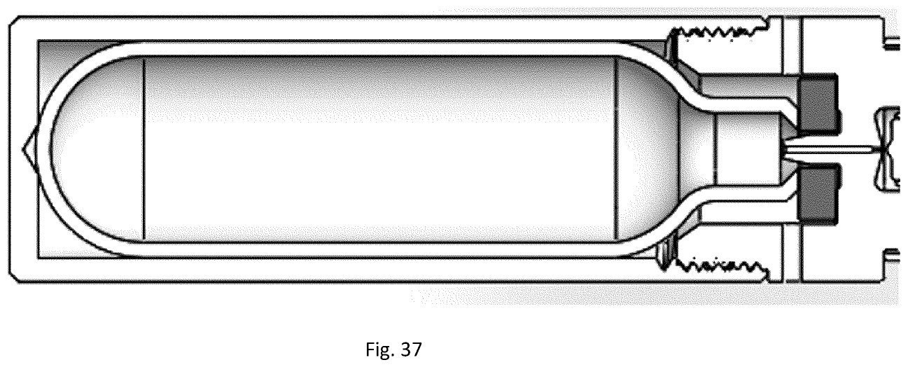

A variety of gas cannisters can be employed for fluid coupling to the regulator 900 and providing the pressurized gas to drive fluid flow. Examples of a thread captured cannister for coupling to the regulator are shown in FIGS. 34-35, and examples of a cap captured cannister for coupling to the regulator are shown in FIGS. 36-37. These gas cannisters can be employed in either the fully automatic mixing device or the semi-automatic mixing device, described in more detail below.

Alternatively, the force to pierce the gas cylinder may be greater than the force required to move the cartridge holder 200 and pierce the drug cartridges 102, 104. In this embodiment, then the fluid pathway will be opened prior to the gas cylinder being pierced. In some embodiments the fluid pathway between the cartridges is opened prior to the gas cylinder. However, since the gas flow is controlled by the solenoids and both are nominally closed until power is provided, in some embodiment the gas cylinder is pierced first.

### Mixing

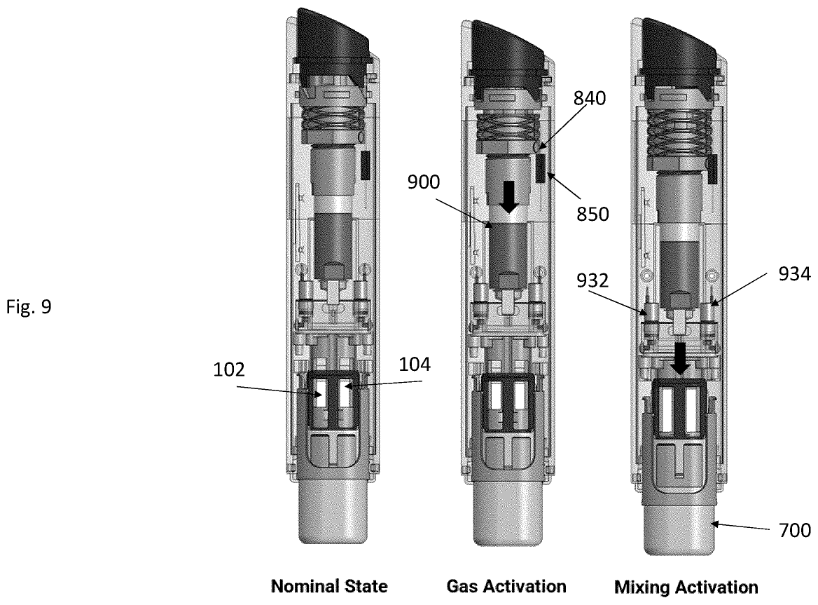

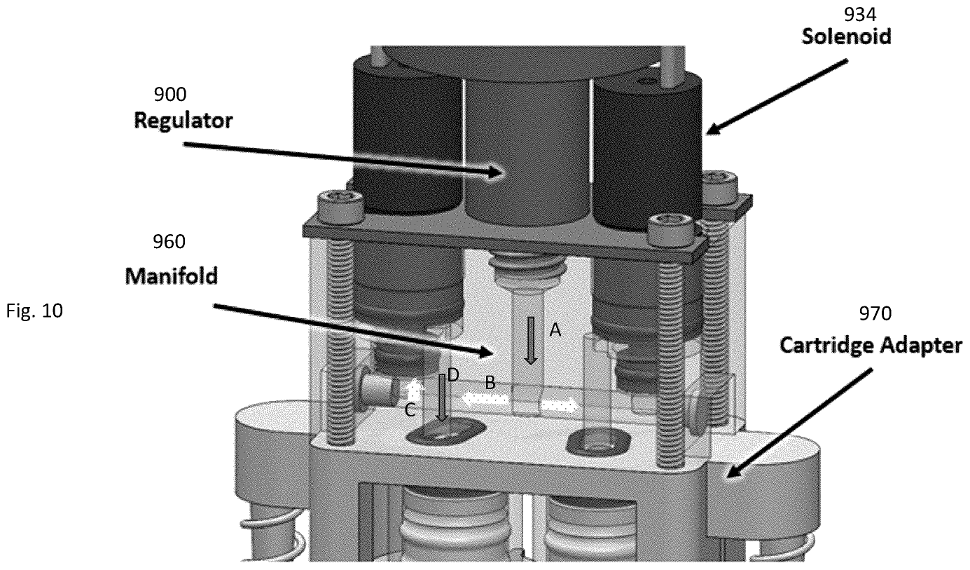

The mixing process is controlled by a plurality (e.g. two) valves. In the exemplary embodiment shown, solenoid valves 932, 934 are employed, which are powered or activated once the power source 840 is brought into proximity with the switch 850, as shown in FIG. 9. The pressurized gas that enters the valves 932, 934 is coming out of the regulator 900 at a specified pressure (which is lower than the pressure within the gas cannister) which can be selected based on the volume and viscosity of the medicaments to be mixed/dispensed by the device. As shown in the close-up view of FIG. 10, the valves 932, 934 are secured in a manifold 960 that has different pneumatic pathways coupled to the valves 932, 934. In the exemplary embodiment shown, a fluid channel extends down from the regulator 900, shown by arrow “A”, and splits into two perpendicular channels, shown by arrow “B”, that are fluidly coupled to the bottom valves 932, 934, to direct the pressurized gas up, shown by arrow “C” into the valves 932, 934, and depending on whether the valves 932, 934 are open or closed, the gas is then routed out of the valve and down, shown by arrow “D” into the cartridges. Thus, in this exemplary embodiment, the gas chamber, regulator, valves and valve manifold all disposed above the drug cartridges 102, 104; however alternative plumbing or channel configurations can be employed as desired, e.g. to minimize the form factor of the device. The manifold 960 is then attached to a cartridge adapter 970 that is used to transfer the air from the manifold 960 to the drug cartridges 102, 104. Where needed, O-rings are used to create seals to prevent gas from leaking.

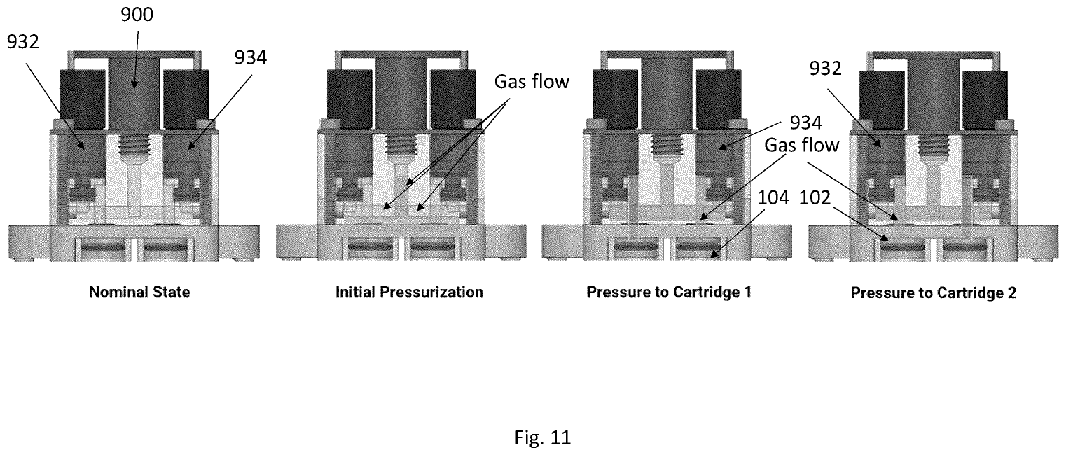

When the gas cylinder is punctured, the manifold 960 is pressurized and sends pressurized gas to both valves 932, 934. In some embodiments, the nominal or initial state for each valve is the “off” position meaning the valves are closed so that pressurized gas is unable to flow through the valves 932, 934 from the inlet gas chamber pressure, as shown in the first illustration of FIG. 11. In the off position, the valve vent is connected to the cartridge allowing any pressure in the drug cartridges to vent to atmosphere through the valves 932, 934.

Once the electrical system is powered up (e.g. the magnet 840 is proximate the switch 850, as shown in FIG. 6-7) the right-hand solenoid valve 904 is given power to change it to the “on” state to open the valve and allow gas pressure to flow through the solenoid valve 934 and into drug cartridge 104. As described above, this pressure pushes the plunger 112 in that cartridge 104 down.

As plunger 112 is depressed downward, the substance (whether active ingredient or diluent) is flowed out of the cartridge 104 and through the mixing channel 310 (as shown in FIG. 2A) and into the adjacent cartridge 102. Consequently, as the volume of the substance (whether active ingredient or diluent) increases in the cartridge 102, the opposing plunger 110 in cartridge 102 rises. In some embodiments, the plungers move in tandem and with the same rate, and range, of motion. As the plunger 110 in cartridge 102 rises, the displaced air is vented through the opposing valve 932 to atmosphere. After a set amount of time, the control board in the electrical system removes power from the right valve 934 and instead powers the left valve 902. This reverses the flow of gas, and in turn, the medicament flows in the reverse direction from the left drug cartridge 102, through the mixing channel 310, and into the right drug cartridge 104. Accordingly, in some embodiments only one valve is powered at time to open and permit gas flow, thereby commencing the mixing of the substances within the two cartridges. Alternatively, in some embodiments each valve is powered simultaneously (e.g. initiation of the nominal state). The regulate can be equipped with a sensor to detect any leakage (e.g. when a valve is in the off/closed state) and trigger an alert (e.g. audible, visual or tactile) to the user, thereby notifying the user of a potential risk of inadequate/undesired mixing and dispensing operation.

It will be apparent to artisans of ordinary skill that although the exemplary embodiments of the present disclosure depict a two-cartridge device, additional cartridges can be included, and each can include a separate valve to permit selective opening of the valve and mixing of the contents of its associated container. For example, a plurality of cartridges (and valves with requisite channels coupled to the cartridges) can be configured in a circular ring (similar to a gun barrel) to provide multiple stages, and substances, for mixing.

### Delivery

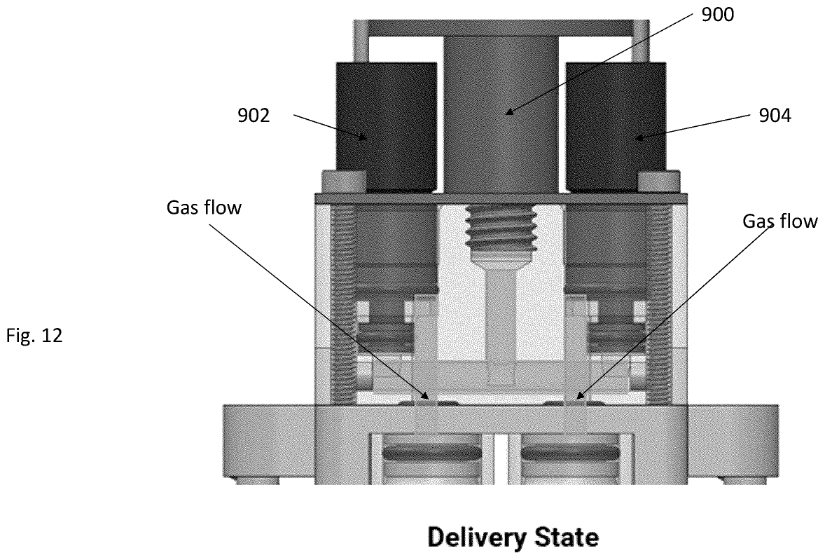

Once the mixing process is completed, the control board powers both valves 932, 934 such that pressure goes to both drug cartridges 102, 104, as shown in FIG. 12. This ensures that once the device is put into its delivery state, the drug is pushed out of both cartridges 102, 104 and through mixing channel 310, through the delivery septum 340, delivery hub 400 and through the delivery needle 600. In some embodiments, it is possible to turn power off to both valves 932, 934 as soon as mixing is complete to conserve battery life. In such embodiments, a limit switch can be used to sense the position of the needle shield 700 and once the needle shield has been fully compressed upwardly, the system powers the valves 932, 934 for delivery.

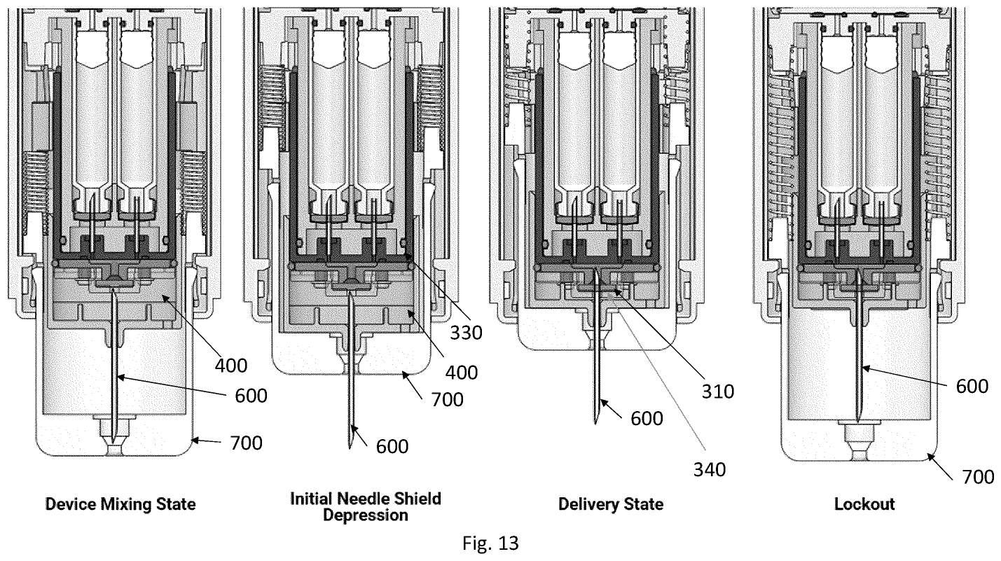

As shown in FIG. 13, to get the device into the delivery state, the user depresses the needle shield 700, which can be done by placing the distal end of the shield against the patient's skin at the desired injection site and pressing the device into the patient (thereby sliding the needle shield upwards). As the needle shield 700 is depressed it contacts the delivery hub 400 of the device (as shown in FIG. 2A). Continued motion of the needle shield 700 pushes the delivery hub 400 upwards relative to the mixing hub 330. This causes the delivery needle 600 to puncture the septum 340 and enter the mixing channel 310. At this point the fluid pathway is open and the drug can be pushed through the delivery needle 600.

### Device Indicators

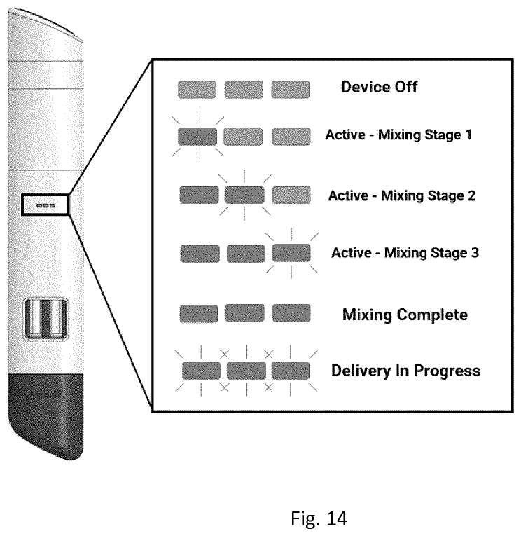

In addition to the view window 1400 that allows the user to visualize the mixing process and delivery of the medicament, the device disclosed herein also has indicia (e.g. multiple LEDs) used to communicate the states of the device to the user. In an exemplary embodiment shown in FIG. 14, when all LEDs are off, the indication is that the device is not powered on.

For purpose of illustration and not limitation, in an exemplary embodiment, once activated, and the mixing process has begun, the LEDs from left to right turn on and flash. If a single LED is flashing, this can convey that the device is indicating that the first third of the mixing process is occurring. During the second third of the mixing process, the first LED can become solid while the middle LED flashes. During the final third of the mixing process, the first and middle LEDs are solid, while the third flashes. Once all LEDs are on and solid, mixing is complete.

To indicate that delivery is in progress, all LEDS can flash. The user can see in the viewing window to determine when the delivery process is complete. Additionally or alternatively, sensors can be incorporated in the device such that the indicator lights communicate to the user that the delivery is completed.

There are many potential alternatives to the LEDs described herein that could be used to communicate the device state to the user. Accordingly, artisans of skill will recognize that myriad of indicia schemes and LED sequences can be employed to convey real time information of the status of any/all step of the operation of the device.

### Needle Shield Lockout

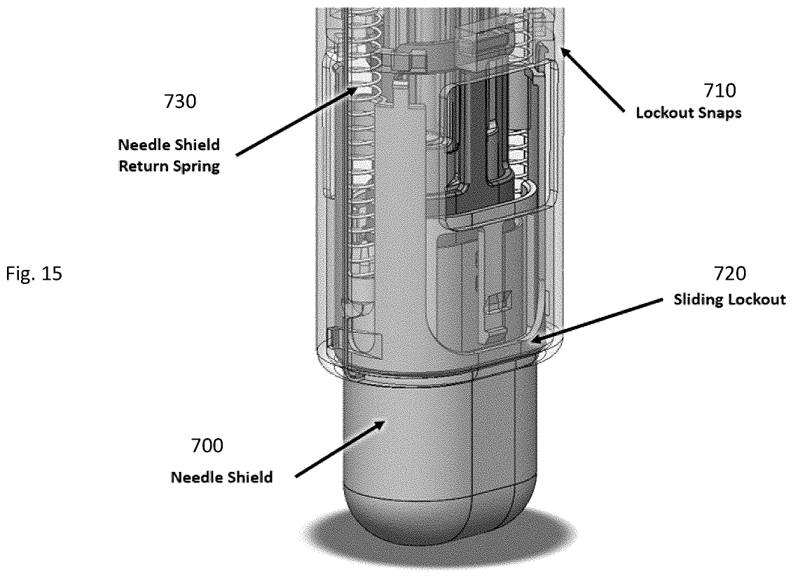

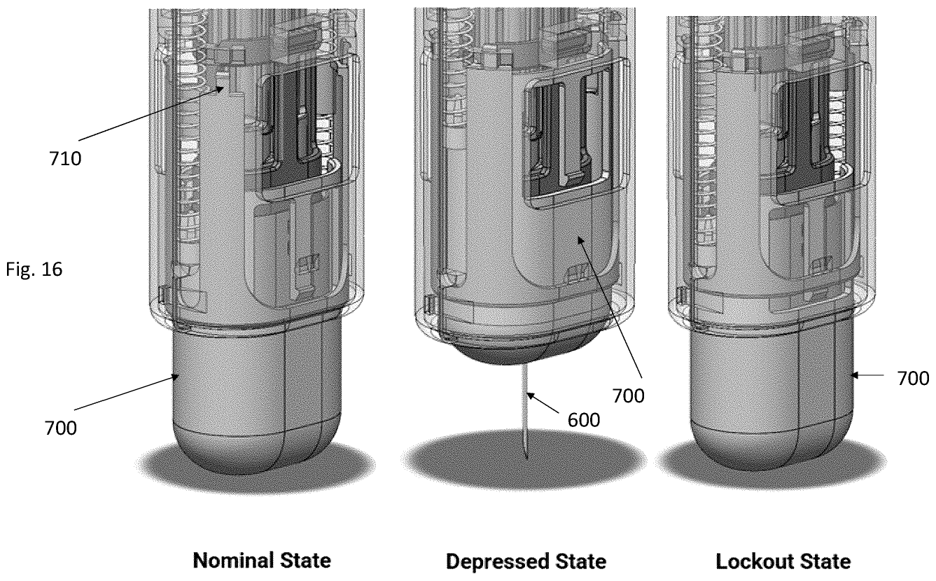

As shown in FIGS. 15-16, the needle shield 700 lockout mechanism utilizes a sliding lockout components 710 and features on the housing ensure the device can only be used once. In its nominal state both the needle shield 700 and the sliding lockout components 710 are in their most distal position, as shown in FIG. 15. As the user depresses the needle shield 700 upwardly, it continues to move until the upper portion of the needle shield 700 contacts a ledge on the upper part of the sliding lockout 720. This contact along with the continued motion of the needle shield 700 pushes the sliding lockout 720 upwards and causes the flanges on the sliding lockout 720 to be displaced above/over and engage with ledges on the housing, as shown in FIG. 16. These protrusions hold the sliding lockout 720 in this upper position and prevent it from returning to its nominal state. Although an exemplary configuration of male/female mechanical union is disclosed for purpose of illustration, additional and alternative structures can be employed to establish the retention of the needle shield as described herein.

As the needle shield 700 is pushed back out by the needle shield 700 return springs 730, a biased arm in the middle of the viewing window on the needle shield 700 is displaced over/above the lower face of the sliding lockout 720. In the nominal state of the device, the sliding lockout 720 is disposed in a lower position inhibiting or prohibiting the biased snap arm to engage with the lockout component. However, at this stage the snap engages with the lower face of the sliding lockout 720 and inhibits/prohibits the needle shield 700 from being depressed again. As the lockout snaps 710 pushes up on the sliding lockout 720, features along the side of the housing prevent the sliding lockout 720 from displacement upward, leading to a locked system.

### Device Electrical System

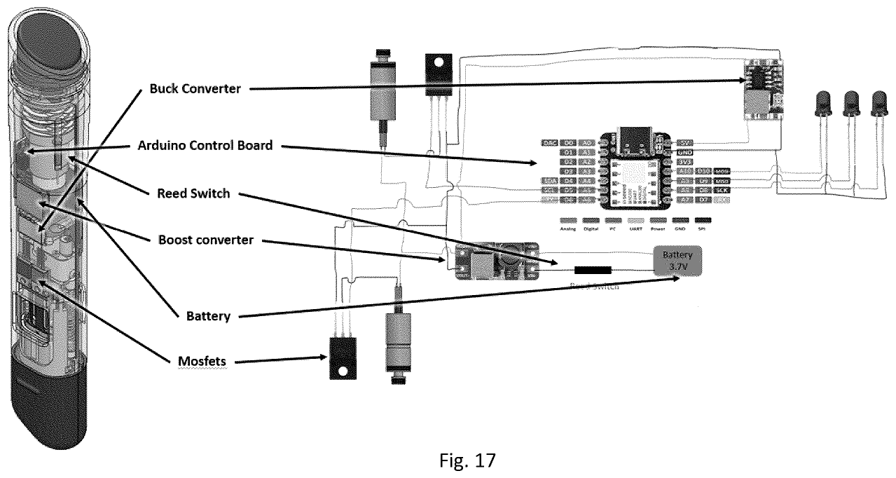

The device disclosed herein can be powered and controlled by an electrical system that is powered on when the user activates the device. In the exemplary embodiment shown in FIG. 17, the power system includes an onboard battery that outputs 3.7V. However, the valves require 12V of power to be activated. In order to achieve this power level, a boost converter can be used to increase the voltage to the required amount. This increased voltage runs to a plurality (e.g. two) metal oxide semiconductor field transistor (MOSFET) and a buck converter. The buck converter takes the increased voltage and reduces it to the appropriate level for the Arduino control board.

The Arduino control board maintains and executes the program that controls the solenoid valve states. The Arduino sends voltage signals to a metal oxide semiconductor field transistor (MOSFET). By varying the voltage signal from the Arduino to the MOSFET, the amount of voltage flowing from the MOSFET to the solenoid valves can be actively controlled. This allows the system to provide either the full 12V of power from the boost converter to the solenoids to turn them on, as well as reduce that voltage to approximately 6V after a short time period to hold the valve in its active state.

### Semi-Automated Mixing and Automated Delivery Device

In accordance with another aspect of the disclosure, a device is provided which can provide semi-automated mixing (rather than the entirely automated mixing described above in connection with the device of FIGS. 1-17) which allows a user to interact with, and control, the mixing process. Once the semi-automated mixing is completed, the delivery of the mixed medicament can be performed automatically (similar to the fully automated, i.e. mixing plus dispensing, device of FIGS. 1-17).

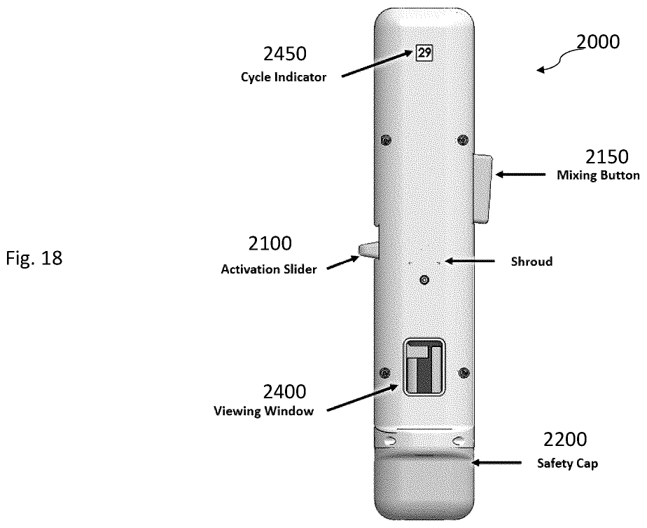

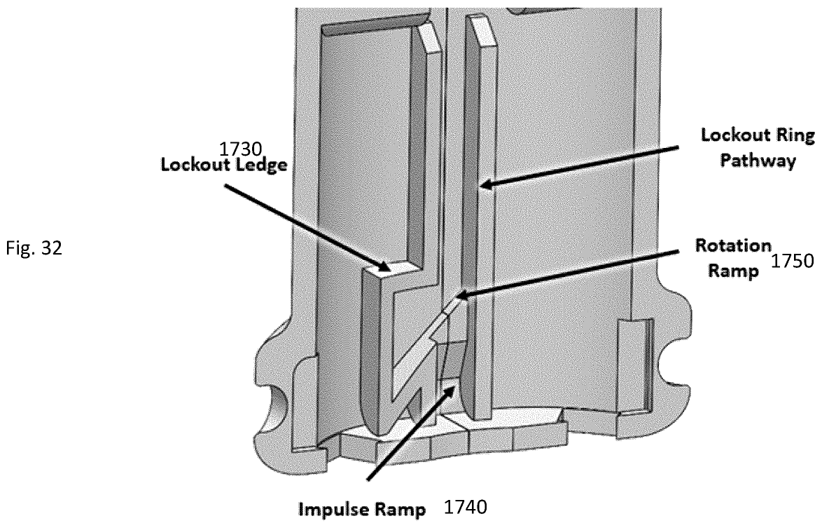

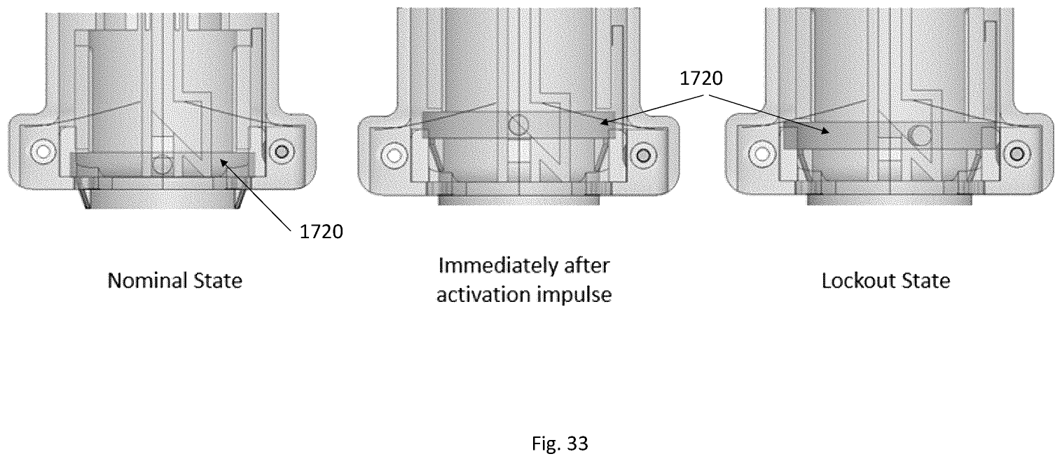

The semi-automated device of FIGS. 18-33 includes the same components shown in FIGS. 1-3 and described above in connection with the fully automated device of FIGS. 1-17, and the entire contents of which are hereby incorporated into the description of the semi-automated device below.

### Device Use States

The device contains 2000 an outer shroud that acts as the main body housing that the user holds and operates during device use. The main user touch points are the activation slider 2100 (or mixing activation mechanism) on the side of the device and the mixing button 2150 disposed, in the exemplary embodiment shown on the opposite side so that a user can operate the activation slider with one (or more) finger of one hand and the mixing button with one (or more) finger of the same hand such that the device is “hand held” and fully operable with a single hand. While using the device, the user can visualize the mixing process through the viewing window 2400 and keep track of the cycles remaining with the cycle Indicator 2450.

The device starts in its nominal state with the activation slider 2100 down and the mixing button 2150 extending outwardly from the exterior of the housing or shroud. The user activates the device by moving the activation slider 2100; in the exemplary embodiment depicted in FIG. 18, the user pushes the activation slider 2100 upwardly and the slider moves within a notch or recess formed in the outer shell of the housing. This operation enables the mixing operation to commence. It will be readily apparent to artisans of skill in the art that the activation slider can be configured to move in additional/alternative directions, and the housing can have a corresponding slot shape to limit the range of motion of the activation slider. This step puts the device into its mixing state and also pierces the compressed gas cylinder to begin powering mixing as described in further detail below.

Upon device activation, compressed gas begins to mix the contents of the drug cartridges within the device and enter the device mixing state. The devices (i.e. fully automated and semi-automated device) disclosed herein are particularly advantageous where the drug(s) contained within the cartridges require or benefit from a number of mixing cycles. A mixing cycle can be defined as depressing and holding the mixing button down until the plunger 110 of the left cartridge 102 reaches the bottom. At that point the user releases the mixing button 2150 and waits until the plunger 112 on the right cartridge 104 reaches the bottom. This process is repeated until a specified number of cycles is reached. Each time the user depresses and releases the mixing button 2150, the cycle indicator 2450 will decrease by 1 digit. This can be particularly helpful to inform a user how many mixing cycles have been performed, and how many more may be required to achieve the desired amount of mixing for a particular active pharmaceutical ingredient and/or diluent. For example, the user can continue to depress the mixing button 2150 to iterate the transfer of contents between drug cartridges 102, 104 and watch the number displayed on the viewing window 2400 decrease until the indicator reaches “0”, signaling the device is ready for delivery.

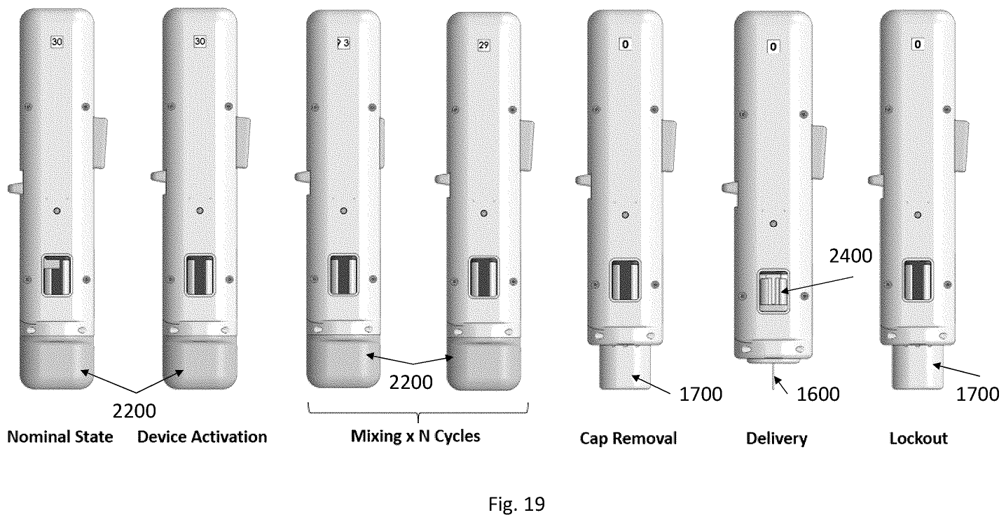

As shown in the various stages of operation of FIG. 19, to deliver the drug to the patient, the user first removes the safety cap 2200 from the distal end of the device. Once removed, the user places the distal end of the needle shield 1700 up against the patient at the injection site and pushes the device towards the patient. This causes the needle shield 1700 to compress and expose the delivery needle 1600. The De delivery needle 1600 enters the patient, and once the needle shield 1700 is fully compressed, the drug will begin to flow through the needle 1600. The user holds the device in this position until they can visually see through the viewing window 2400 that plungers in both cartridges are at the bottom of the cartridges.

Once drug delivery is complete, the user pulls the device away from the patient and the needle shield 1700 automatically extends downward to enclose the needle 1600. The device is now in the lockout state, with the needle shield 1700 mechanism now preventing the needle shield 1700 from being depressed again and exposing a used needle.

### Device Activation

To allow for extremely viscous drugs or solutions that require many mixing cycles, the mixing and delivery process is powered by compressed gas cylinder. Compressed gas is beneficial as the mixing/dispensing driving force since it allows for a large amount of potential energy to be stored in a relatively small space.

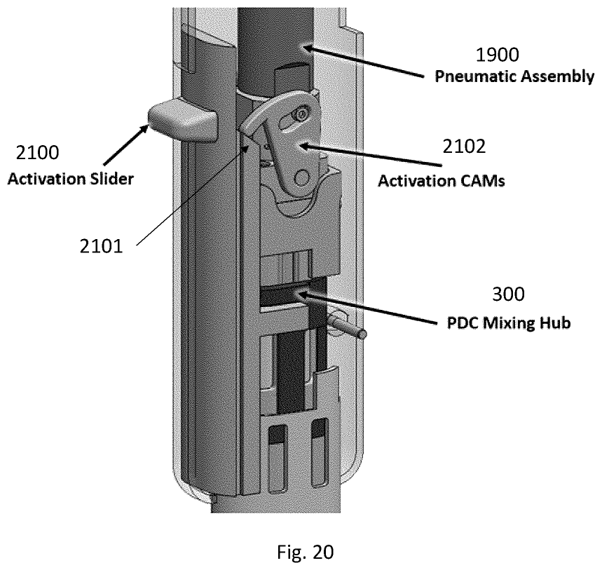

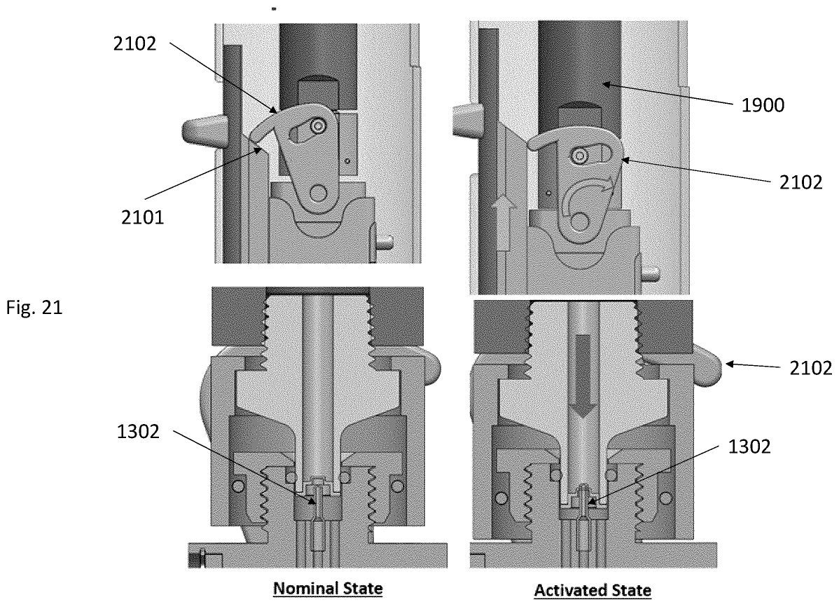

To activate the semi-automatic mixing device, the device is moved from its nominal state to its mixing state, and the gas cylinder is pierced or otherwise opened to begin driving the flow of fluids within the device. This is achieved by pushing up on the slider 2100 on the side of the device. In the exemplary embodiment shown in FIGS. 20-21, the activation slider 2100 has a fork feature (on the interior of the housing) that extends around the mixing hub 300 of the device to couple the two components together. As the activation slider 2100 moves (e.g. translates vertically upward), the mixing hub 300 moves in tandem causing the mixing needles 302, 304 (as described in connection with FIGS. 1-3) located within the mixing hub 300 to pierce the drug cartridges 102, 104 held in the stationary cartridge holder 200. It is also contemplated in another embodiment (not shown) that the cartridges could be prefilled syringes.

As shown in FIGS. 20-21, the activation slider 2100 has a ramp feature(s) 2101 extending on the interior of the housing that interfaces with the activation cams 2102. These cams rotate as the activation slider 2100 moves vertically. As the cams 2102 rotate, a pathway in the cams 2102 pulls down on the pneumatic assembly 1900. This causes the pneumatic assembly 1900 to be driven down onto a piercing member 1302 (e.g. spike or needle), which breaks a seal and releases the gas. In some embodiments, the pathway in the activation cams 2102 has a flat feature towards the end of travel so that any backpressure from the gas cannister is held in place without requiring the user to maintain a force on the activation slider 2100. In operation, the relative dimensions of the activation slider travel distance and range of rotation of the activation cam is configured to provide a timing or sequence such that the mixing pathway is opened prior to the gas being released from the canister.

### Mixing

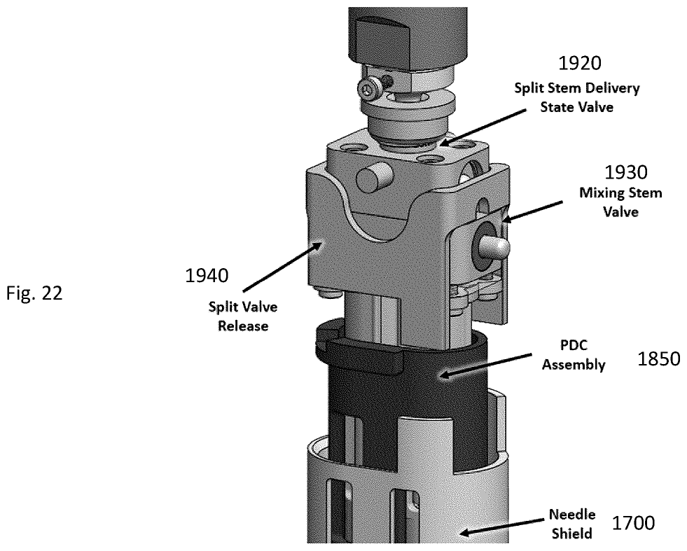

To drive mixing and delivery of the drug, compressed gas passes through the semi-automated mixing and automated delivery valve system. This system includes a plurality of valves that operate to direct the compressed gas through multiple directions within the device. In the exemplary embodiments shown in FIGS. 22-27, the mixing system includes a delivery state valve 1920, a mixing stem valve 1930 and a split release valve 1940. In some embodiments, the valves are oriented vertically with the split stem valve 1920 disposed above and in abutting contact with the mixing stem valve 1930. Both the split stem valve 1920 and mixing stem valve include multiple channels traversing through the respective valve from the top surface through the bottom surface for directing compressed gas therethrough. In some embodiments the split valve release 1940 is configured to surround at least a portion of the mixing stem valve 1930 and split stem delivery state valve 1920, and move (e.g. translate vertically) along an outer surface of the mixing stem valve 1930 and split stem delivery state valve 1920.

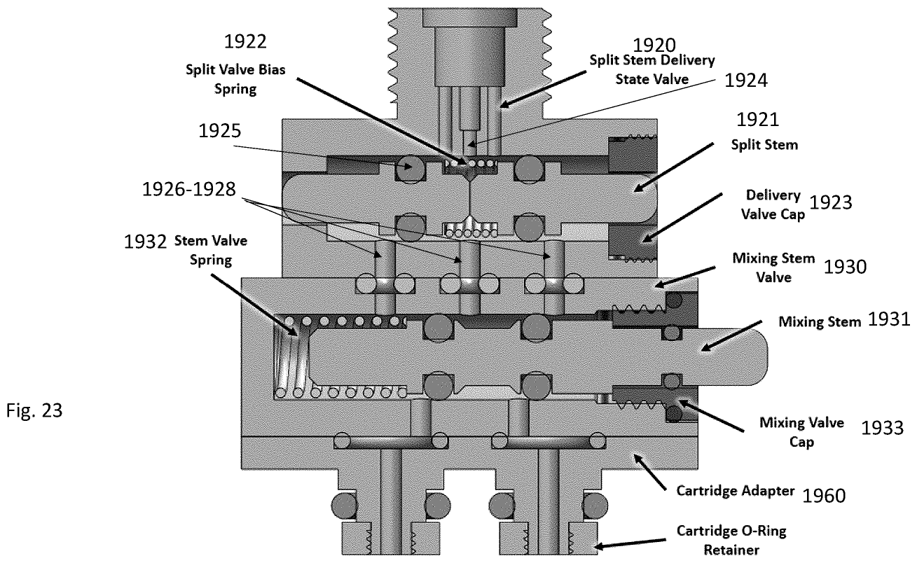

The delivery state valve 1920 includes a split stem valve that is operated (e.g. pushed into an open position) by both gas pressure and a mechanical spring. In the exemplary embodiment shown in FIG. 23, delivery state valve 1920 includes a spring 1922 which can bias the split stem 1921 laterally between open and closed configurations. Also, the pressurized gas can be delivered through inlet 1924. O-rings 1925 can be included within the seats of the split stem 1921 to maintain a seal and direct all the pressurized gas through one, or more, of the outlets 1926, 1927, 1928 of the split stem delivery state valve. The split stem 1921 is held in place by the split valve release 1940.

The mixing stem valve 1930 includes a single mixing stem that is biased to its outer position by a stem valve spring 1932. The delivery valve 1920 can be coupled (e.g. mechanically attached via bolts) to the mixing stem valve 1930 from the top and the primary drug container 1850 and cartridge adapter 1960 are coupled (e.g. mechanically attached via bolts) to the mixing stem valve 1930 from the bottom. The outlets of the split stem delivery state valve 1926, 1927, 1928 are vertically aligned with the inlets of the mixing stem valve 1930 to form a continuous channel for directing pressurized gas. All pneumatic pathways are sealed by using O-rings in compression and nested in seats defined by upright protrusions (e.g. see structure abutting each side of O-ring 1925 in FIG. 23) of the valve stems.

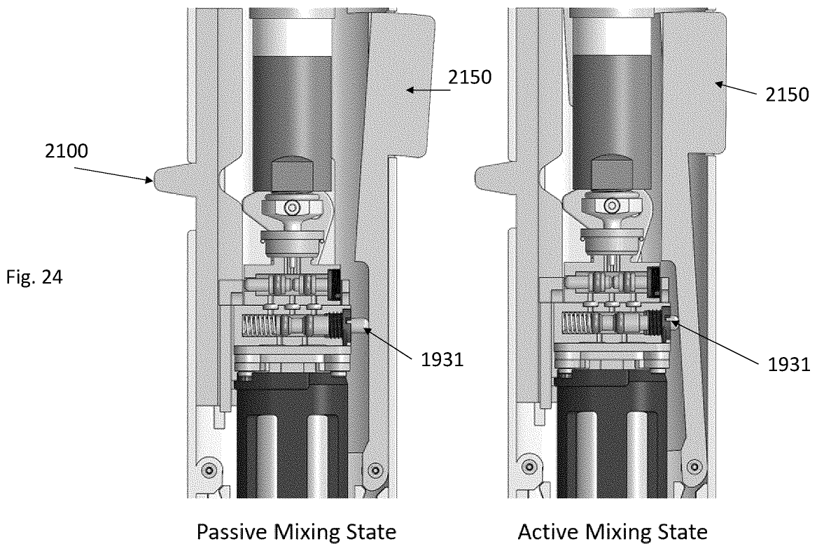

The user can control mixing by depressing and releasing the mixing button 2150. The mixing button 2150 directly interfaces with the mixing stem 1931 in the mixing stem valve 1930. In the exemplary embodiment shown in FIG. 24, the lever arm of mixing button 2150 extends downward to engage/abut the distal end of the mixing stem 1931. In the passive mixing state, the user is applying no force to the mixing button. In this condition, the stem valve spring 1932 displaces the mixing stem 1931 to its outer position and in turn holds the mixing button 2150 in its outer position (i.e. projecting outwardly from the device housing). When the user pushes on the mixing button 2150 and overcomes the stem valve spring 1932 force, the mixing stem 1931 shifts to its inner position and in turn changes the direction of the gas flow, thereby entering the active mixing state.

Gas flows through the regulator and into the delivery state valve 1920. During the mixing state, the spilt valve release 1940 is in its lower position, preventing the split stems 1921 from moving outwards. This forces the gas down through the middle orifice 1927 and into the mixing stem valve 1930. The mixing stem valve 1930 controls which cartridge 102, 104 receives pressure from the compressed gas cylinder and which cartridge is venting to atmosphere. With the mixing stem 1931 in its outer position, gas flows down through the central orifice 1927 of the mixing stem valve 1930 and is directed to the right output orifice 1938. This pathway leads to the cartridge adapter 1960 that connects the valving system to the drug cartridges 102, 104 in the primary drug container. The gas pressure pushes down on the plunger 112 in the cartridge 104 and the O-Ring seals on the cartridge adapter 1960 prevent any gas leaks during use. As the plunger 112 of one container 104 is pushed down, the fluid path connection in the primary drug container causes the opposing plunger 110 in the adjacent cartridge 102 to be pushed upward. The non-pressurized air above that plunger flows up through the cartridge adapter 1960, mixing stem valve 1930, and delivery state valve 1920 until it vents to atmosphere.

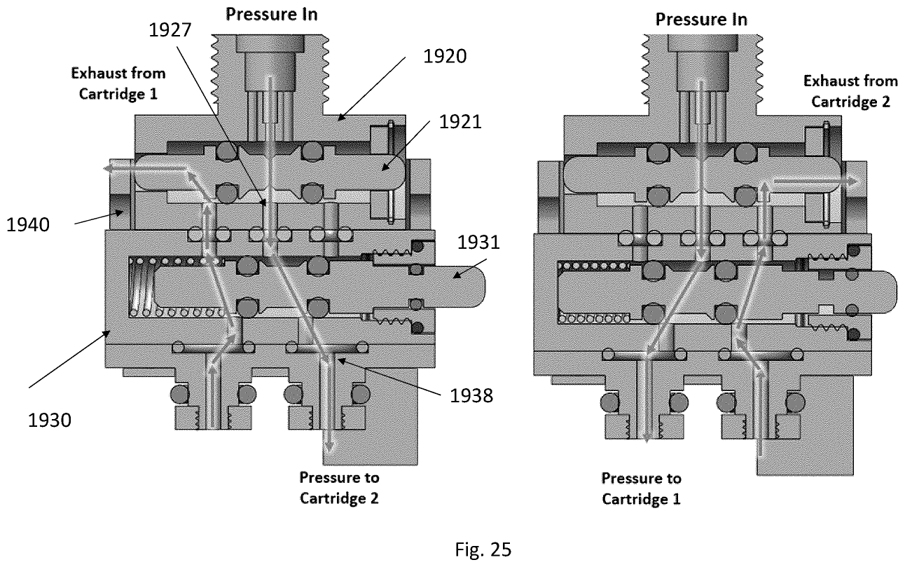

After pressurized gas is routed along a first fluid path through the valve(s) to the drug container as shown on the left side of FIG. 25, the process can be switched or iterated to adjust the split stem 1931 position and route the pressurized gas along a second fluid path through the valve(s) to the second drug container as shown in the right side of FIG. 25. Thus the valve(s) disclosed herein multidirectional and configured to alternate the flow of gas within the valve and route the gas to the desired drug container to depress the plunger therein, and in turn transfer the contents of that container through the mixing channel and into the adjacent drug container. In some embodiments, during the mixing state (with the split valve release 1940 in the first position), the pressurized gas is only routed through a single channel in the valve(s) to provide pressurized gas to only one drug container at a time.

In some embodiments, the valve design of the semi-automatic mixing device depicted in FIG. 23 can also be employed in the fully automatic mixing device of FIG. 1.

### Delivery

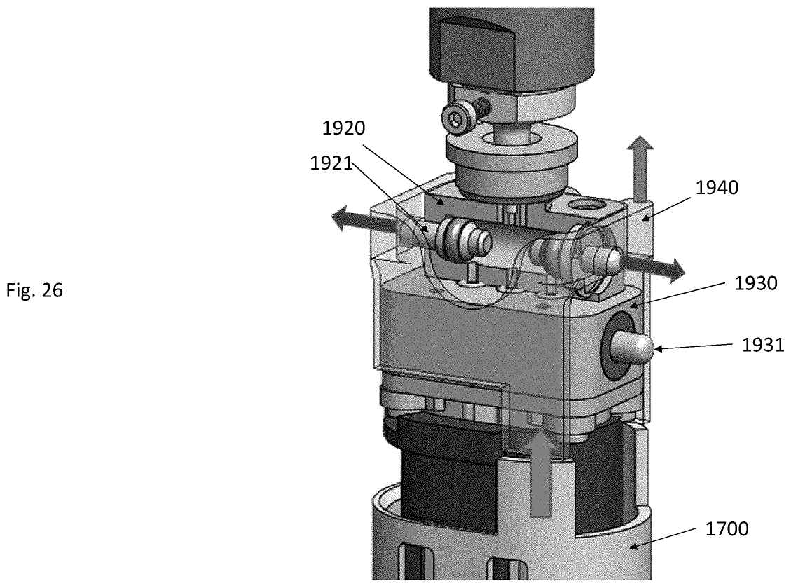

Drug delivery is achieved by changing the state of the delivery stem valve. As described above, the user pushes the needle shield 1700 against the patient to push the needle 1600 into the skin and start the drug delivery process. Internally, the needle shield 1700 contacts the split valve release 1940 and pushes it upwards, as shown in FIG. 26. This motion releases the split stem 1921 inside the delivery state valve 1920. The two split stems 1921 separate and are pushed outwards by both pressure and a spring 1922 until they reach a hard stop internal to the delivery state valve.

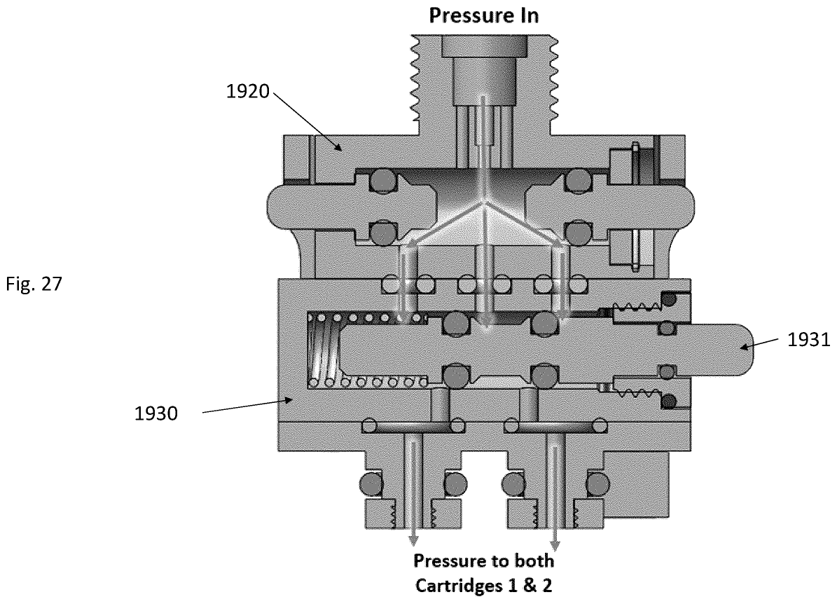

With the split stems 1921 in their outer position, what used to be ports used for ventilation 1926-1928 are now used as pressurization ports to the mixing stem valve 1930, as shown in FIG. 27. This motion now provides pressure to both drug cartridges 102, 104, simultaneously, regardless of the position of the mixing stem 1931 in the mixing stem valve 1930.

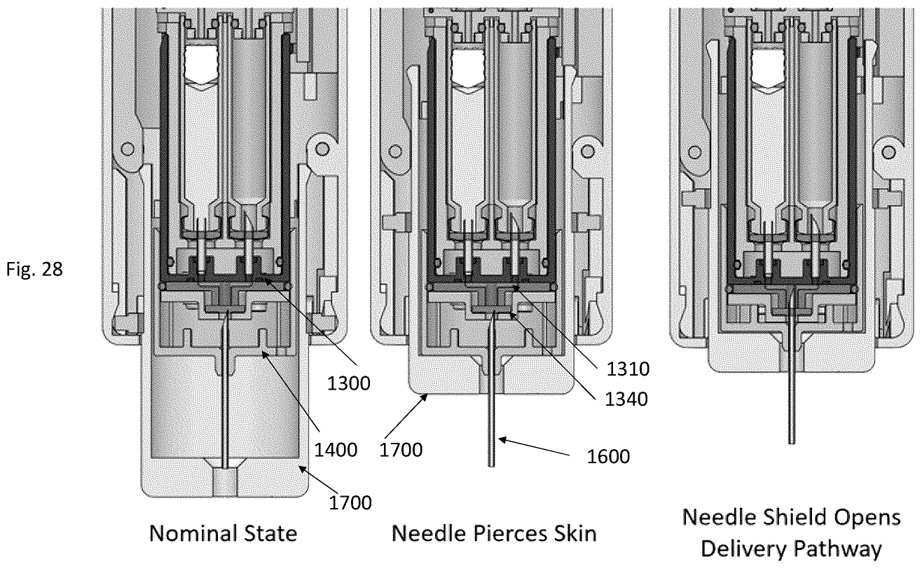

As shown in the exemplary embodiment of FIG. 28, to open the fluid pathway and allow the drug to flow through the delivery needle 1600, the delivery needle 1600 punctures the septum 1340 in the primary drug container and enters the mixing channel 1310. This is done automatically during the delivery process when the needle shield 1700 is compressed upwardly. As the needle shield 1700 retracts into the device, it contacts the distal end of the delivery hub 1400 and pushes it upwards along the mixing hub 1300. The motion continues until the delivery hub 1400 contacts the mixing hub 1300. The previously mixed contents of the drug containers 102, 104 can then be dispensed through the mixing channel 1310 and through the delivery needle 1600 and into he patient.

### Cycle Count Indicator

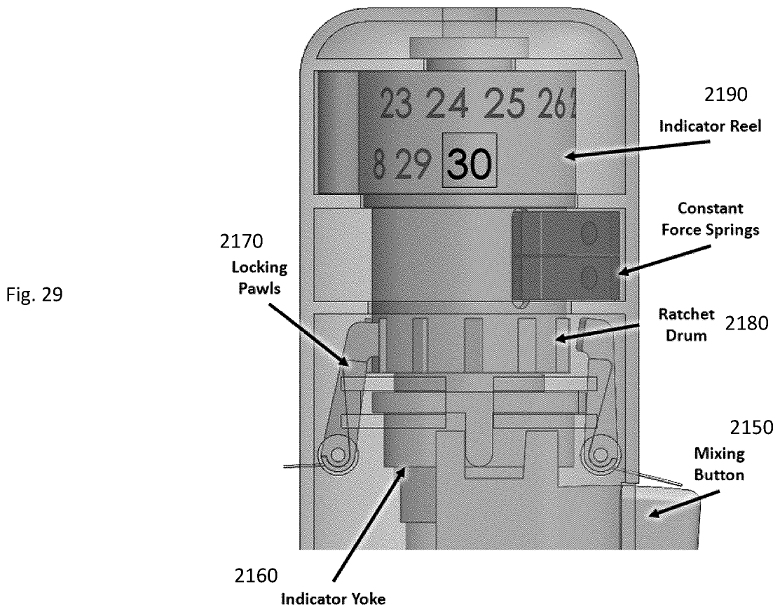

As shown in FIG. 29, throughout the mixing process, the mixing button 2150 can also drive the ratcheting of the cycle indicator mechanism in addition to the valve system. This conveys to the user the number of mixing cycles performed, and/or remaining, and aligns the associated number which is printed on an internal indicator reel 2190 to align with the cycle indicator window 2450 for user viewing. In an exemplary embodiment, the top of the mixing button 2150 has a fork feature that interfaces with the indicator yoke 2160. When the mixing button 2150 is depressed and released it shifts the indicator yoke 2160 back and forth within a pathway in the shroud. At each of the indicator yoke 2160 extreme positions, it pushes on a locking pawl 2170. The two locking pawls, which are biased against the ratchet drum 2180 with torsion springs, work together to prevent the ratchet drum 2180 from rotating due to the force applied to it by the constant force springs. In some embodiments, the torsion springs could be replaced by plastic spring features built into the pawls.

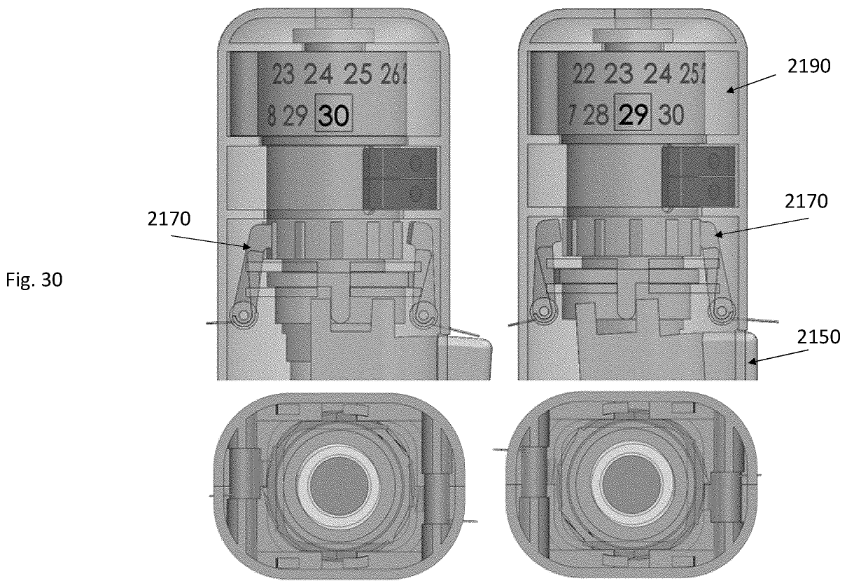

As shown in FIG. 30, the ratchet drum 2180 includes a plurality of teeth equally distributed around the circumference of the drum. At any given time, a locking pawl 2170 is engaged with one of the teeth to prevent rotation of the ratchet drum 2180. A single cycle, consists of one locking pawl 2170 releasing the drum while the other locking pawl 2170 catches or engages the drum, and then the second locking pawl releases the drum (i.e. is pivoted away from the drum), and the original locking pawl catching the next circumferentially-adjacent tooth. A pair of locking pawls 2170 are offset such that the ratchet drum 2180 rotates a half step as one unlocks and the other locks.

A reel 2190 is wrapped around the top portion of the ratchet drum 2180 so that numbers can be displayed to the user. As the ratchet drum 2180 rotates, the numbers on the real decrease until it reaches 0. Once at 0, the user knows that they have completed the desired number of mixing cycles.

### Needle Shield Lockout

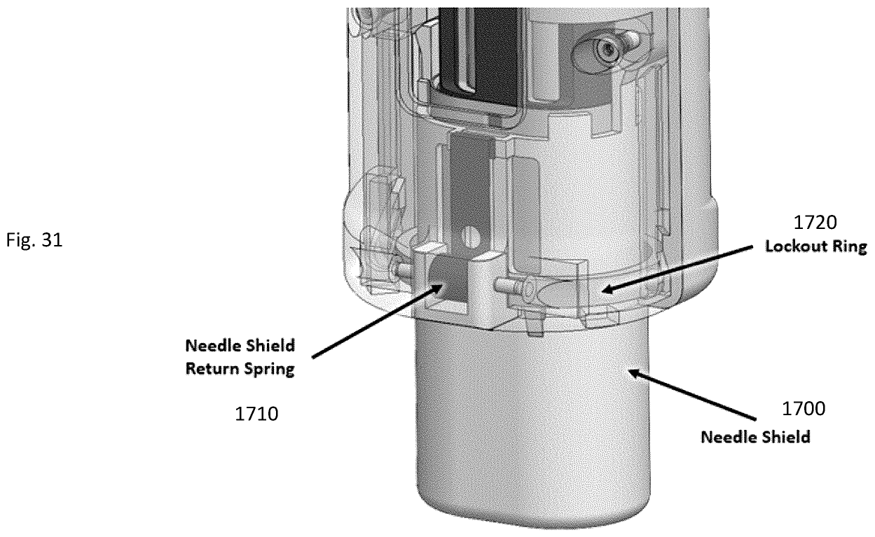

In accordance with another aspect of the disclosure, the needle shield 1700 prevents visualization of the needle 1600 prior to, during, and after use of the device. Additionally, the lockout mechanisms ensures that after drug delivery is completed, the used delivery needle 1600 cannot be accessed again. In the exemplary embodiment shown in FIGS. 31-33, this is done by utilizing a lockout ring 1720 that surrounds the needle shield 1700 and has small bosses that follow a pathway in the shroud. However, alternative structures, e.g. lockout ring which does not extend around the entire circumference, can be employed if so desired.

As the user depresses the needle shield 1700 against the patient's body, the protrusions on the lockout ring 1720 initially cause resistance due to a ramp 1740 in the pathway. The entire lockout ring 1720 flexes or is temporarily displaced laterally in order to allow the lockout ring 1720 and needle shield 1700 to get past this point. The user overcomes this resistance to depress the needle shield 1700 further. This is done to create a large load that once overcome, causes the user to collapse the entire needle shield 1700 without hesitation.

Once compressed, a constant force spring pulls on the needle shield 1700 to return it to its nominal position. However, the protrusions on the lockout ring 1720 are unable to get past the initial ramp 1740 that they snapped over previously. Instead, the surface is angled by ridge 1750 causing the protrusions and lockout ring 1720 to rotate about the center of the needle shield 1700 until the protrusions are under the lockout ledge 1730. At this point if the user goes to depress the needle shield 1700 again, lockout ring would be displaced directly upward and abut the lockout ledge 1730, which has a internally extending flange which prevents any further vertical motion of the needle shield 1700 that would expose the delivery needle 1600.

While the disclosed subject matter is described herein in terms of certain preferred embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

1. A mixing and drug delivery system comprising:
a housing configured to hold a first container and a second container, wherein the first container contains a first medicament component and the second container contains a second medicament component;
a first seal associated with the first container;
a second seal associated with the second container;
a fluid communication assembly having a fluidic channel between the first container and the second container, the fluid communication assembly configured to be displaced from a first position to a second position within the housing thereby opening, removing or otherwise piercing the first seal and second seal to provide a fluidic pathway between the first container and the second container;
a mixing system configured to alternately transfer the first and second medicaments between the first and second containers during a mixing phase;
a pressurized gas chamber at least partially disposed in the housing to pressurize the mixing system;
a mixing activation mechanism, the mixing activation mechanism displacing the pressurized gas chamber from a first position to a second position to open or otherwise pierce the pressurized gas chamber;
at least one valve configured to release a portion of pressurized gas that facilitates the transfer of the first and second medicaments components between the first and second containers by the mixing system, wherein the transfer between first and second containers causes the first and second medicament components to become a mixed medicament; and
a needle delivery assembly configured to be in fluid communication with the first and second containers during a delivery phase.

2. The mixing and drug delivery system of claim 1, wherein the mixing activation mechanism is coupled to a power source, the power source displaced from a first position to a second position to activate the at least one valve.

3. The mixing and drug delivery system of claim 2, wherein the power source controls operation of the at least one valve.

4. The mixing and drug delivery system of claim 2, wherein the power source is a magnet.

5. The mixing and drug delivery system of claim 1, wherein the at least one valve includes two solenoid valves, a first solenoid valve in fluid communication with the first container and a second solenoid valve in fluid communication with the second container.

6. The mixing and drug delivery system of claim 5, wherein only one solenoid valve is powered at a time.

7. The mixing and drug delivery system of claim 6, wherein the first and second solenoid valves are powered simultaneously.

8. The mixing and drug delivery system of claim 5, wherein the first and second solenoid valves are initially in a closed position.

9. The mixing and drug delivery system of claim 1, wherein the pressurized gas chamber is disposed above the first container and a second container.

10. The mixing and drug delivery system of claim 1, wherein the mixing system further comprises a first gas-driven plunger associated with the first container and a second gas-driven plunger associated with the second container.

11. A mixing and drug delivery system comprising:
a housing configured to hold a first container and a second container, wherein the first container contains a first medicament component and the second container contains a second medicament component;
a first seal associated with the first container;
a second seal associated with the second container;
a fluid communication assembly having a fluidic channel between the first container and the second container, the fluid communication assembly configured to be displaced from a first position to a second position within the housing thereby opening, removing or otherwise piercing the first seal and second seal to provide a fluidic pathway between the first container and the second container;
a mixing system configured to alternately transfer the first and second medicaments between the first and second containers during a mixing phase;
a pressurized gas chamber at least partially disposed in the housing to pressurize the mixing system;
a mixing activation mechanism, the mixing activation mechanism displacing the pressurized gas chamber from a first position to a second position to open or otherwise pierce the pressurized gas chamber;
at least one valve configured to release a portion of pressurized gas that facilitates the transfer of the first and second medicaments components between the first and second containers by the mixing system, wherein the transfer between first and second containers causes the first and second medicament components to become a mixed medicament;
a mixing button, the mixing button movable between a first position and a second position to open and close the at least one valve; and
a needle delivery assembly configured to be in fluid communication with the first and second containers during a delivery phase.

12. The mixing and drug delivery system of claim 11, wherein the mixing system further comprises a first fluid path from the pressurized gas chamber through the valve to the first container, and a second fluid path from the pressurized gas chamber through the valve to the second container.

13. The mixing and drug delivery system of claim 11, wherein the mixing system further comprises a first gas-driven plunger associated with the first container and a second gas-driven plunger associated with the second container.

14. The mixing and drug delivery system of claim 13, wherein the at least one valve includes a multi-directional valve configured to alternate the flow of gas directed to the first and second gas-driven plungers based on user input to the mixing button.

15. The mixing and drug delivery system of claim 14, whereupon receiving the user input to the mixing button causes the mixing system to drive the first gas-driven plunger to transfer the first medicament component from the first container into the second container with the second medicament component.

16. The mixing and drug delivery system of claim 13, wherein displacement of the first gas-driven plunger downward in the first container causes displacement of the second gas-driven plunger upwards in the second container.

17. The mixing and drug delivery system of claim 14, further comprising a valve release component coupled to the multi-directional valve, valve release component displaced from a first position to a second position within the housing.

18. The mixing and drug delivery system of claim 17, wherein pressurized gas flows to only one of the first plunger or second plunger at a time when the valve release component is in the first position.

19. The mixing and drug delivery system of claim 17, wherein pressurized gas flows to both the first plunger and the second plunger simultaneously when the valve release component is in the second position.

20. The mixing and drug delivery system of claim 11, further including a delivery seal configured to prevent fluid communication between the fluidic channel and the needle during a mixing phase.
